(19) United States
(12) Patent Application Publication
(10) Pub. No.: US 2003/0229132 A1
(43) Pub. Date: Dec. 11, 2003

(54) Small molecule modulators of apoptosis

(76) Inventors: Jack Nguyen, South San Francisco, CA (US); Jim Wells, Burlingame, CA (US)

Correspondence Address: Choate, Hall & Stewart, Exchange Place, 53 State Street, Boston, MA, 02109, US

(21) Appl. No.: 10/364,440

(22) Filed: Feb. 11, 2003

Related U.S. Application Data
(60) Provisional application No. 60/405,822, filed on Aug. 23, 2002.
(60) Provisional application No. 60/356,488, filed on Feb. 12, 2002.

(51) Int. Cl. C12Q 1/68; A61K 31/404; C07D 209/02; C 07D 4 3/02; C07D 209/36

(52) U.S. Cl. 514/414; 435/6; 514/418; 548/465; 548/485

(57) ABSTRACT

The present invention provides methods of identifying modulators of apoptosis. Additionally provided are methods of contacting a cell with a compound capable of decreasing the amount of cyto c necessary to form apoptosome, and thereby inducing apoptosis in

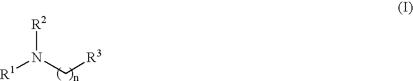

and pharmaceutically acceptable derivatives thereof, wherein R¹, R², R³ and n are as described generally and in classes and subclasses herein, and additionally provides pharmaceutically compositions thereof, and methods for the use thereof as modulators of apoptosis and for the treatment of disorders caused by excessive or insufficient apoptotic activity.

### RELATED APPLICATIONS

This application asserts priority to Provisional Application No. 60/356,488, filed Feb. 12, 2002 and No. 60/405,822, filed Aug. 23, 2002, each of which is incorporated herein by reference in its entirety.

### BACKGROUND OF THE INVENTION

Apoptosis, which is a programmed cell-suicide mechanism, plays a significant role in a variety of normal processes including, but not limited to, immune system education (e.g., elimination of autoreactive cells), viral defense (altruistic cell suicide may deny viral replication within a host) and tissue homeostasis (ensuring an appropriate balance of cell production vs. cell eradication). Any disruption of this process, either by inappropriate triggering of apoptosis or by impairment of apoptosis, can contribute to the development or progression of many diseases. For example, it is believed that either too little or too much cell death contributes to half of the main medical illnesses for which adequate therapy or prevention is lacking (for example, cancer, autoimmunity, restenosis, persistent infections, ischemia, heart failure, neurodegeneration, inflammation, osteoarthritis, human immunodeficiency virus, bacterial infection, allograft rejection and graft versus host disease, Type I diabetes and trauma).

Because of the significance of apoptosis in the development and progression of disease, many approaches have been developed in an effort to regulate apoptosis. For example, many drugs (chemotherapeutic and others) activate apoptosis as a function of their activity by either inducing the expression of pro-apoptotic genes or by downregulating the expression of anti-apoptotic genes. Upon the induction of apoptosis, cytochrome c (cyto c) translocates from the mitochondria to the cytosol, where it forms a large oligomeric complex with Apaf-1 and procaspase-9 (see, P. Li et al., Cell 91, 479-89 (1997)). Within this complex, called the apoptosome, caspase-9 becomes activated by proteolytic cleavage and proceeds to activate downstream caspases, ultimately leading to full implementation of the apoptotic program (see, E. A. Slee et al., J Cell Biol 144, 281-92 (1999)). Since controlling gene expression is a relatively early event in the apoptosis cascade, many tumors become resistant to chemotherapy by mutating or overexpressing downstream genes. More recently, efforts have been made to identify compounds that target factors further downstream in the apoptotic pathway; the majority of these efforts have focused on identifying inhibitors of anti-apoptotic members of the Bcl-2 family, Bcl-2 and Bcl-X_{L} (see, I. J. Enyedy et al., J Med Chem 44, 4313-24 (2001); J. L. Wang et al., Proc Natl Acad Sci USA 97, 7124-9. (2000); A. Degterev et al., Nat Cell Biol 3, 173-82 (2001)). Both proteins are normally anchored to the mitochondrial membrane and function to inhibit cyto c translocation (see, J. M. Adams, S. Cory, Science 281, 1322-6 (1998)). Thus, compounds that inhibit Bcl-2 or BCl-X_{L} activity enable the release of cyto c into the cytosol and thereby induce apoptosis. To date, however, no small molecule apoptosis activators that function subsequent to cyto c release have been identified.

Clearly, there remains a need for the development of novel therapeutic strategies for modulating the key molecules responsible for regulating apoptosis in cells. In particular, it would be desirable to develop therapeutics capable of selectively targeting (e.g., activating or inhibiting) factors further downstream in the apoptotic pathway.

### BRIEF DESCRIPTION OF THE DRAWINGS

FIG. 1 depicts the identification of small molecules that modulate apoptosis. (A) Approximately 3500 compounds were screened for their ability to inhibit or activate caspase activity in a cell-free apoptosis assay. Compounds that activate caspase activity by the fluorescence screen were subjected to a secondary screen to directly visualize caspase-3 processing. Active compounds from the secondary screen were then resynthesized as purified compounds and rescreened in both assays. (B) Sample plate from the primary fluorescence screen. Each curve represents an individual compound. The DMSO control (closed squares) with vehicle only and the negative control (open squares) with no added cyto c are shown. Compounds having no effect on DEVDase activity are shown as gray curves. An activator is shown as closed diamonds and two inhibitors are shown as open diamonds and closed triangles. (C) A sample immunoblot for the large subunit of caspase-3 from the secondary screen. The negative control has no cyto c, and the DMSO control is the vehicle only. Lanes with (*) are identified as activators, whereas the lane with (#) is an inhibitor. (D) Chemical structures of compounds 1-5.

FIG. 2 depicts activity of compounds 1-5 in cell lysates. (A) Compound activation is cyto c dependent. Cyto c was titrated into S-100 cytoplasmic extracts with vehicle alone or 20 μM compound and procaspase-3 processing was assayed by capture ELISA. (B) Compound activation is dose-dependent. Compounds or vehicle were titrated into S-100 cytoplasmic extracts at a cyto c concentration of 1.25 μM and procaspases-3 processing was assayed by capture ELISA. (C) Immunoblot assay of compound-induced caspase activation. Samples at the 20 μM compound concentration in (B) were separated on SDS-polyacrylamide gel and transferred to polyvinyl membrane. The same membrane was cut and probed with antibodies to caspase-9, the large subunit of active caspase-3, and P13 kinase as a loading control. (D) Compounds act upstream of caspase-9 activation. Cyto c was titrated into caspase-3 immunodepleted extracts with or without 20 μM compounds and assayed by immunoblot for procaspase-9 processing.

FIG. 3 depicts activity of compounds 1-5 in whole cells. Jurkat cells were incubated with the DMSO vehicle, 1 μM staurosporin, or 50 μM compound for 6 hr and then lysed. Samples were then probed by immunoblot for (A) caspase-3 activation or (B) cleavage of PARP. (C) DNA fragmentation analysis. Jurkat cells were incubated with vehicle, 1 μM staurosporin, or 50 μM compound for 8 hr and then lysed. The DNA was isolated by phenol/chloroform extraction, separated on a 2% agarose gel and visualized by ethidium bromide staining. Cell viability assay. Cells were incubated with different concentrations of compound, staurosporin, or vehicle for 22 hr and assayed for viability by MTT test. (D) Jurkat cells were incubated with varying concentrations of compounds 1-5 or vehicle. (E-F) Normal (PBL) and cancer (Jurkat, Molt-4, CCRF-CEM) lymphocyte cell lines were incubated with varying concentrations of compound 2 or staurosporin.

FIG. 4 shows sensitivity of cell lines from the NCI cancer panel to compound 2. Cells were exposed to serial dilutions of compound 2 continuously for 6 days, and cell growth relative to controls was determined by staining with sulforhodamine B. Some cell lines were excluded for clarity. (A) Dose-response plots for leukemia, melanoma, renal cancer and CNS cancer. (B) Dose-response plots for lung, breast and colon cancers.

FIG. 5 shows the Apaf-1 dependence of the activity of compound 2. (A) Jurkat cells were transfected with 20 nM Apaf-1 siRNA for 48 hr and half the cells were lysed and probed with antibodies to Apaf-1, caspase-9, and caspase-3. The other half of the cells was incubated with varying concentrations of (B), compound 2 or (C), Fas ligand for 24 hr and assayed for viability by MTT test. (D) SK-OV-3 cells were transiently transfected with Apaf-1 or vector control for 24 hr and then incubated with vary concentrations of compound 2 and assayed for viability. (E) Cyto c was titrated into SK-OV-3 cell lysate in the presence of 300 μM dATP, with or without 150 nM purified Apaf-1 and 20 μM compound 2 as indicated.

FIG. 6 depicts activity of compounds 1-5 in purified system. (A) Reconstitution of caspase-3 activation using purified components. Reactions containing 31 nM procaspase-3, with or without 4 μM procaspase-9, 160 nM Apaf-1, 5 μM cyto c, and 1 mM dATP as indicated, were assayed by capture ELISA for procaspase-3 processing. (B) Compound activation is cyto c dependent. Cyto c was titrated into reactions containing procaspase-3, procaspase-9, Apaf-1, and dATP, with or without 20 μM compounds. Procaspase-3 processing was assayed by capture ELISA. (C) Compound dose-response curves. Compounds were titrated into reactions containing procaspase-3, procaspase-9, Apaf-1, dATP, and 0.15 μM cyto c and procaspase-3 processing was assayed by capture ELISA.

FIG. 7 depicts activity of compounds 1-5 at low cyto c concentrations in a purified system. Apaf-1 was incubated with vehicle, cyto c, or cyto c plus 20 μM compound as indicated, and then separated on a Superose 6 gel filtration column. Individual fractions were assayed for relative Apaf-1 concentrations by capture ELISA (bar graph), or the ability to activate procaspase-3 processing in lysates (line graph). The percent of Apaf-1 in apoptosomes was determined by dividing the amount of Apaf-1 in fractions 8-11 by the total amount of Apaf-1. The extent of caspase-3 activation (given in arbitrary units) corresponds to the area under the curve for fractions 8-11 in each panel. Black bars represent fractions used for calculations.

### DESCRIPTION OF CERTAIN PREFERRED EMBODIMENTS OF THE INVENTION

In one aspect, the present invention provides modulators of apoptosis. In certain embodiments, the inventive compounds are activators of apoptosis and are useful for the treatment of disorders resulting from insufficient apoptotic activity. In certain other embodiments, the compounds promote the activation of caspases at reduced levels of cyto c. In still other embodiments, the compounds activate caspase-9 and caspase-3 by promoting oligomerization of Apaf-1.

1) General Description of Compounds of the Invention

The compounds of the invention include compounds of the general formula (I) as further defined below:

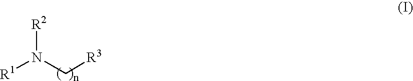

and pharmaceutically acceptable derivatives thereof;

wherein n is 0, 1 or 2;

R¹ is a moiety having the structure

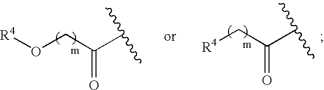

R² is hydrogen, or an aliphatic, heteroaliphatic, aryl, or heteroaryl moiety, or is —(C═O)R⁵; or R¹ and R² taken together are a cycloaliphatic, cycloheteroaliphatic, aryl or heteroaryl moiety;

R³ is an aryl or heteroaryl moiety;

each occurrence of R⁴ is independently an aliphatic, heteroaliphatic, aryl or heteroaryl moiety;

each occurrence of m is independently 0, 1 or 2; and

each occurrence of R⁵ is independently an aliphatic, heteroaliphatic, aryl, or heteroaryl moiety, or is OR⁶, NR⁶R⁷, or SR⁶, wherein each occurrence of R⁶ and R⁷ is independently hydrogen, a protecting group, or an aliphatic, heteroaliphatic, aryl or heteroaryl moiety,

whereby each of the foregoing aliphatic and heteroaliphatic moieties are independently substituted or unsubstituted, linear or branched or cyclic or acyclic, and whereby each of the foregoing cycloaliphatic, cycloheteroaliphatic, aryl and heteroaryl moieties are independently substituted or unsubstituted.

It will be appreciated that for compounds as generally described above, certain classes of compounds are of special interest. For example, one class of compounds of special interest includes those compounds as described generally above and herein, in which R¹ is

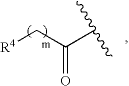

R² is hydrogen and the compound has the structure:

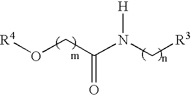

and R³ is a substituted or unsubstituted aryl or heteroaryl moiety, and R⁴, m and n are as described generally herein.

In certain embodiments of special interest, R³ is an aryl or heteroaryl moiety having the structure:

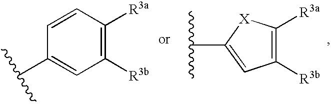

wherein X is O, S, NH or CH₂ and each occurrence of R^{3a} and R^{3b} is independently hydrogen, halogen, substituted or unsubstituted alkyl, cyano, OR^{3c}, SR^{3c}, or NR^{3c}R^{3d}, wherein each occurrence of R^{3c} and R^{3d} is independently hydrogen, protecting group, or an aliphatic, heteroaliphatic, aryl or heteroaryl moiety.

In certain other embodiments of special interest, R⁴ is a cycloaliphatic, heterocycloaliphatic, aryl, or heteroaryl moiety, optionally linked via an alkyl moiety. In certain embodiments, R⁴ is a cycloaliphatic, heterocycloaliphatic, aryl or heteroaryl moiety having the general structure:

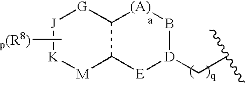

wherein the dotted line represents a single or double bond; A is —CR^{A}—, C(R^{A})₂, 0, S, N, NR^{A}, or C═O; a is 0 or 1; B is —CR^{B}—, C(R^{B})₂, O, S, N, NR^{B}, or C═O; D is C or CH, E is —CR^{E}—, C(R^{E})₂, O, S, N, NR^{E}, or C═O; G is absent or is —CR^{G}, C(R^{G})₂, O, S, N, NR^{G}, or C═O; J is absent or is CR^{J}, C(R^{J})₂, O, S, N, NR^{J}, or C═O; K is absent or is —CR^{K}, C(R^{K})₂, O, S, N, NR^{K}, or C═O; M is absent or is —CR^{M}, C(R^{M})₂, O, S, N, NR^{M}, or C═O; and adjacent members of A, B, D, E, G, J, K and M, if present, are connected by a single or double bond; R⁸ is hydrogen, halogen, or an aliphatic, heteroaliphatic, aryl or heteroaryl moiety and q is 0-1.

Another class of compounds of special interest consists of compounds in which R¹ and R², when taken together are a substituted or unsubstituted monocyclic or bicyclic moiety, n is 1 or 2, and the compound has the structure:

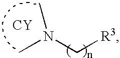

wherein R³ is an aryl or heteroaryl moiety as described generally and in certain subsets herein.

In certain embodiments of special interest, R³ is an aryl or heteroaryl moiety having the structure:

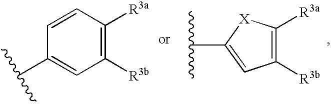

wherein X is O, S, NH, or CH₂, and each occurrence of R^{3a} and R^{3b} is independently hydrogen, halogen, substituted or unsubstituted alkyl, cyano, OR^{3c}, SR^{3c}, or NR^{3c}R^{3d}, wherein each occurrence of R^{3c} and R^{3d} is independently hydrogen, protecting group, or an aliphatic, heteroaliphatic, aryl or heteroaryl moiety.

In certain embodiments, R¹ and R² taken together form cyclic compounds having the general structure:

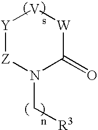

where n and R³ are as defined above, W is —NR^{W}, CR^{W}, O, S or C═O; V is NR^{V}, CR^{V}, O, S or C═O; s is 0 or 1; Z is —NR^{Z}, CR^{Z}, O, S or C═O; and Y is —NR^{Y}, CR^{Y}, O, S or C═O, and wherein R^{W}, R^{V}, R^{Y} and R^{Z} are each independently hydrogen, or an aliphatic, heteroaliphatic, aryl or heteroaryl moiety, or any two of R^{W}, R^{V}, R^{Y} and R^{Z}, taken together form a substituted or unsubstituted aryl or heteroaryl moiety.

In certain embodiments of special interest, R¹ and R², taken together form cyclic compounds having the following structures:

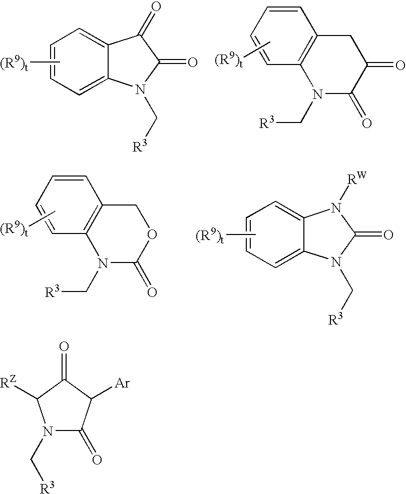

wherein R³, R^{W} and R^{Z} are as described generally above, and wherein R⁹ is hydrogen, halogen, or an aliphatic, heteroaliphatic, aryl or heteroaryl moiety and t is an integer from 0-4.

Another class of compounds of special interest consists of compounds in which R¹ is

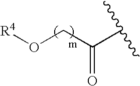

and the compound has the structure:

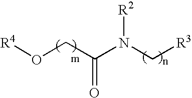

wherein R⁴ is lower alkyl; m is 0 or 1; n is 0, 1 or 2; R² is an aliphatic, heteroaliphatic, aryl, or heteroaryl moiety, or is —(C═O)R⁵; each occurrence of R⁵ is independently hydrogen or an aliphatic, heteroaliphatic, aryl, or heteroaryl moiety, or is OR⁶, NR⁶R⁷, or SR⁶, wherein each occurrence of R⁶ and R⁷ is independently hydrogen, a protecting group, or an aliphatic, heteroaliphatic, aryl or heteroaryl moiety; and R³ is a substituted or unsubstituted aryl or heteroaryl moiety.

In certain embodiments, R³ is an aryl or heteroaryl moiety having the structure:

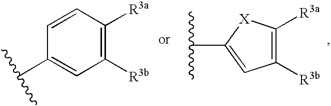

wherein X is O, S, NH or CH₂, and each occurrence of R^{3a} and R^{3b} is independently hydrogen, halogen, substituted or unsubstituted alkyl, cyano, OR^{3c}, SR^{3c}, or NR^{3c}R^{3d}, wherein each occurrence of R^{3c} and R^{3d} is independently hydrogen, protecting group, or an aliphatic, heteroaliphatic, aryl or heteroaryl moiety.

A number of important subclasses of each of the foregoing classes deserve separate mention; these subclasses include subclasses of the foregoing classes in which:

i) R¹ is

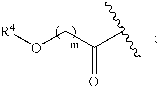

ii) m is 0 or 1;

iii) R⁴ is lower alkyl;

iv) R⁴ is a cycloalipahtic, cycloheteroaliphatic, aryl or heteroaryl moiety having the structure:

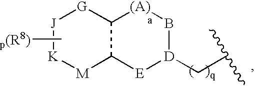

as described generally and in classes and subclasses herein;

v) R⁴ is a cycloaliphatic, cycloheteroaliphatic, aryl or heteroaryl moiety having one of the structures:

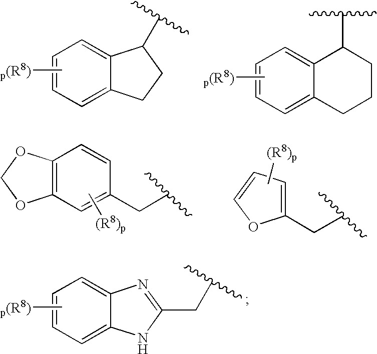

vi) R⁴ is a cycloaliphatic, cycloheteroaliphatic, aryl or heteroaryl moiety having one of the structures:

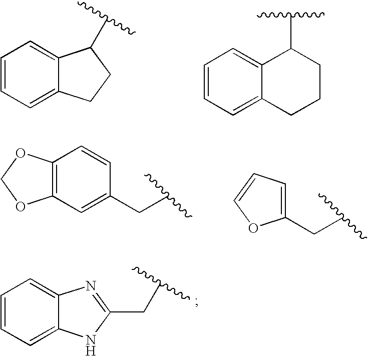

vii) n is 0, 1 or 2;

viii) R³ is a substituted aryl or heteroaryl moiety having the structure:

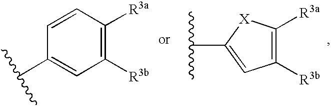

wherein X is O, S, NH, or CH₂, and each occurrence of R^{3a} and R^{3b} is independently hydrogen, halogen, substituted or unsubstituted alkyl, cyano, OR^{3c}, SR^{3c}, or NR^{3c}R^{3d}, wherein each occurrence of R^{3c} and R^{3d} is independently hydrogen, protecting group, or an aliphatic, heteroaliphatic, aryl or heteroaryl moiety;

ix) R³ is a substituted aryl moiety having the structure:

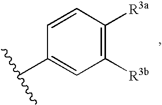

wherein each occurrence of R^{3a} and R^{3b} is hydrogen, halogen or substituted or unsubstituted alkyl;

x) R^{3a} and R^{3b} are each Cl, Br or CF₃;

xi) in certain embodiments, compounds G, H, I, J, K and L, as described in the exemplification herein, are excluded from compounds as described generically above and in subclasses herein;

xii) in certain embodiments, compounds G, H, I, J, K and L, as described in the exemplification herein, are provided as pharmaceutical compositions comprising a therapeutically effective amount of any one of the compounds, and a pharmaceutically acceptable carrier and optionally further comprising an additional therapeutic agent;

xiii) in certain embodiments, compounds G, H, I, J, K and L, as described in the exemplification herein, are useful as modulators of apoptosis, and in certain embodiments are useful as inducers of apoptosis, and, as such are useful for the treatment of disorders resulting from insufficient apoptotic response; and

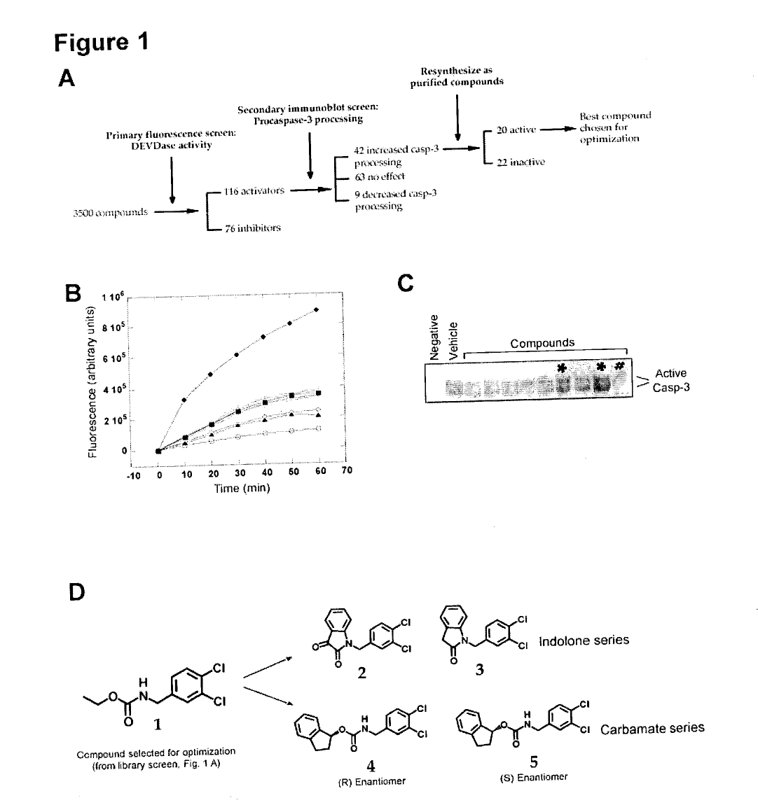

xiv) in certain embodiments, compounds 1, 2, 3 and 5, as described in FIG. 1 herein, are useful as modulators of apoptosis, and in certain embodiments are useful as inducers of apoptosis, and, as such are useful for the treatment of disorders resulting from insufficient apoptotic response.

As the reader will appreciate, compounds of particular interest include, among others, those which share the attributes of one or more of the foregoing subclasses. Some of those subclasses are illustrated by the following sorts of compounds:

1) Compounds of the Formula:

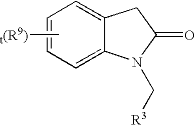

wherein R³ is a substituted aryl or heteroaryl moiety having the structure:

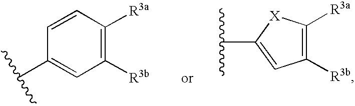

wherein X is O, S, NH, or CH₂, and each occurrence of R^{3a} and R^{3b} is independently hydrogen, halogen, substituted or unsubstituted alkyl, cyano, OR^{3c}, SR^{3c}, or NR^{3c}R^{3d}, wherein each occurrence of R^{3c} and R^{3d} is independently hydrogen, protecting group, or an aliphatic, heteroaliphatic, aryl or heteroaryl moiety; R⁹ is hydrogen, halogen, or an aliphatic, heteroaliphatic, aryl or heteroaryl moiety; and t is an integer from 0-4.

In certain embodiments, compounds having the following structure are provided:

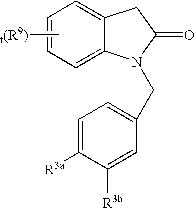

wherein R^{3a}, R^{3b}, R⁹ and t are as described generally above and in subclasses herein. In certain embodiments of special interest, R^{3a} and R^{3b} are each a halogen or a substituted or unsubstituted alkyl. In certain other embodiments of special interest, R^{3a} and R^{3b} are each Cl, Br or CF₃. In yet other embodiments of special interest, each occurrence of R⁹ is hydrogen.

II) Compounds of the Formula:

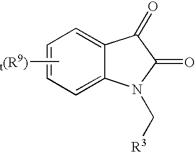

wherein R³ is a substituted aryl or heteroaryl moiety having the structure:

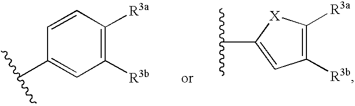

wherein X is O, S, NH, or CH₂, and each occurrence of R^{3a} and R^{3b} is independently hydrogen, halogen, substituted or unsubstituted alkyl, cyano, OR^{3c}, SR^{3c}, or NR^{3c}R^{3d}, wherein each occurrence of R^{3c} and R^{3d} is independently hydrogen, protecting group, or an aliphatic, heteroaliphatic, aryl or heteroaryl moiety; R⁹ is hydrogen, halogen, or an aliphatic, heteroaliphatic, aryl or heteroaryl moiety; and t is an integer from 0-4.

In certain embodiments, compounds having the following structure are provided:

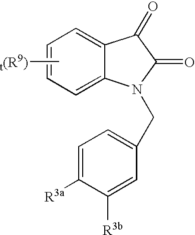

wherein R^{3a}, R^{3b}, R⁹ and t are as described generally above and in subclasses herein. In certain embodiments of special interest, R^{3a} and R^{3b} are each a halogen or a substituted or unsubstituted alkyl. In certain other embodiments of special interest, R^{3a} and R^{3b} are each Cl, Br or CF₃. In yet other embodiments of special interest, each occurrence of R⁹ is hydrogen.

III) Compounds of the Formula:

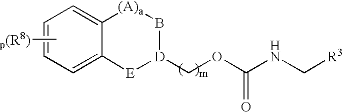

wherein m is O or 1; A is CR^{A}, C(R^{A})₂, O, S, N, NR or C═O; a is 0 or 1; B is —CR^{B}—, C(R^{B})₂, O, S, N, NR^{B}, or C═O; D is C or CH, E is —CR^{E}—, C(R^{E})₂, O, S, N, NR^{E}, or C═O, and A, B, D, and E are connected by a single or double bond; R⁸ is hydrogen, halogen, or an aliphatic, heteroaliphatic, aryl or heteroaryl moiety; p is 0-1; and R³ is a substituted aryl or heteroaryl moiety having the structure:

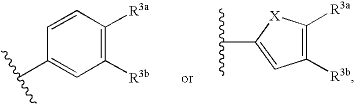

wherein X is O, S, NH, or CH₂, and each occurrence of R^{3a} and R^{3b} is independently hydrogen, halogen, substituted or unsubstituted alkyl, cyano, OR^{3c}, SR^{3c}, or NR^{3c}R^{3d}, wherein each occurrence of R^{3c} and R^{3d} is independently hydrogen, protecting group, or an aliphatic, heteroaliphatic, aryl or heteroaryl moiety.

In certain embodiments, compounds having the following structure are provided:

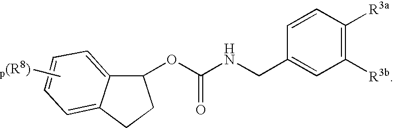

In certain embodiments, R^{3a} and R^{3b} are each halogen or substituted or unsubstituted alkyl. In certain other embodiments, R^{3a} and R^{3b} are each Cl, Br or CF₃. In still other embodiments, compounds depicted above are the S enantiomer.

Some of the foregoing compounds can exist in various isomeric forms, e.g., stereoisomers and/or diastereomers. Furthermore, certain compounds, as described herein may have one or more double bonds that can exist as either the Z or E isomer, unless otherwise indicated. The invention additionally encompasses the compounds as individual isomers (e.g., as either the R or S enantiomer) substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds of this invention which are of particular interest include those which:

exhibit the ability to modulate apoptosis;

exhibit the ability to activate apoptosis;

exhibit the ability to promote oligomerization of Apaf-1; and/or

exhibit cytotoxic or growth inhibitory effect on cancer cell lines maintained in vitro or in animal studies using a scientifically acceptable cancer cell xenograft model.

As discussed above, certain of the compounds as described herein exhibit activity generally as modulators of apoptosis. More specifically, compounds of the invention demonstrate activity as activators of apoptosis and thus, in one aspect, the invention further provides a method for treating disorders resulting from insufficient apoptosis (e.g., cancer, autoimmune diseases, restenosis, and persistent infections); for a general discussion of apoptosis and apoptosis-based therapies, see, Reed, J. Nature Reviews Drug Discovery2, 111-121 (2002). As discussed above, many drugs (chemotherapeutic and others) activate apoptosis as a function of their activity by either inducing the expression of pro-apoptotic genes or by downregulating the expression of anti-apoptotic genes (see, R. W. Johnstone, A. A. Ruefli, S. W. Lowe, Cell 108, 153-164 (2002)). Since controlling gene expression is a relatively early event in the apoptosis cascade, many tumors become resistant to chemotherapy by mutating or overexpressing downstream genes (see, R. W. Johnstone, A. A. Ruefli, S. W. Lowe, Cell 108, 153-164 (2002)). Without wishing to be bound by any particular theory, the compounds of the invention unexpectedly appear to induce apoptosis specifically by a novel mechanism of action, whereby the compounds promote the oligomerization of Apaf-1 to a greater extent than would be seen in the absence of the compounds.

In another aspect of the present invention, methods for identifying modulators of apoptosis are provided. The methods involve: a) combining in a first mixture at least Apaf-1, cyto c, and a hydrolyzable nuceloside phosphate; b) measuring a first extent of oligomerization; c) combining in a second mixture the same compoments as in the first mixture plus a test compound; d) measuring a second extent of oligomerization; and e) comparing the first extent of oligomerization with the second extent of oligomerization to determine whether the test compound is a modulator of apoptosis. In certain embodiments, the methods are adapted to identify apoptosis activators. In certain other embodiments, the methods are adapted to identify apoptosis inhibitors.

In another aspect of the invention, a process is provided for inducing apoptosis in a cell. The process comprises contacting a cell capable of forming an active apoptosome, the apoptosome comprising cyto c and Apaf-1, with a compound capable of decreasing the amount of cyto c necessary to form the active apoptosome, and thereby inducing apoptosis in the cell. In one embodiment, the active apoptosome additionally comprises Procaspase-9. In another embodiment, the process additionally comprises contacting with an agent to increase the level of Apaf-1 within the cell. In another embodiment, the agent to increase the level of Apaf-1 in the cell is a DNA methyltransferase inhibitor or an Apaf-1 expression vector.

In another aspect of the invention another process is provided for inducing apoptosis in a cell. The process comprises contacting a cell with a compound that promotes cyto c-dependent oligomerization of Apaf-1, thereby inducing a caspase cascade and apoptosis in the cell, In one embodiment, the process further comprises contacting the cell with an agent that increases cellular levels of Apaf-1 protein or Procaspase-9 protein.

In one embodiment of the aforementioned processes, the cell is a human cell. In another embodiment, the human cell is a peripheral blood lymphocyte, a MCF 10A cell, a human mammary epithelial cell, a human umbilical vein endothelial cell, or a prostate epithelial cell. In another embodiment of the aforementioned processes, the cell is a cancer cell. In another embodiment, the cell is a human cancer cell. In another embodiment, the human cancer cell is a hematopoietic cancer cell, a skin cancer cell, a colon cancer cell, a breast cancer cell, a lung cancer cell, a renal cancer cell, a CNS cancer cell, an ovarian cancer cell or a prostate cancer cell. In another embodiment, the human cancer cell is a leukemia cell, a lymphoma cell or a melanoma cell.

In another embodiment of the aforementioned processes, the human cancer cell is located within a solid tumor or is located on the surface of a solid tumor. In another embodiment, the human cancer cell is in vitro. In another embodiment, the in vitro human cancer cell is a Jurkhat cell, a Molt-4 cell, a CCRF-CEM cell, a RPMI-8226 cell, a LOX IMVI cell, a BT-549 cell, a NCI/ADR-RES cell, a MDA-MB 435 cell, an HCC-2998 cell, or a NCI—H23 cell.

In yet another aspect of the present invention, methods for using the inventive compounds are provided. The methods generally involve the administration of a therapeutically effective amount of the compound or a pharmaceutically acceptable derivative thereof to a subject (including, but not limited to a human or animal) in need of it. In certain embodiments, the inventive compounds are useful for the treatment of autoimmune diseases, restenosis, and persistent infections. In certain other embodiments, the compounds are useful for treatment of cancers, particularly apoptosis-sensitive cancers such as many types of leukemia.

2) Compounds and Definitions

As discussed above, in certain embodiments, this invention provides novel compounds with a range of biological properties. In particular, compounds of this invention have biological activities relevant for the treatment of disorders caused by insufficient or excessive apoptotic activity. In certain embodiments of special interest, compounds of the invention have biological activities relevant for the treatment of disorders caused by insufficient apoptotic activity.

Compounds of this invention include those specifically set forth above and described herein, and are illustrated in part by the various classes, subgenera and species disclosed elsewhere herein.

It will be appreciated by one of ordinary skill in the art that asymmetric centers may exist in the compounds of the present invention. Thus, inventive compounds and pharmaceutical compositions thereof may be in the form of an individual enantiomer, diastereomer or geometric isomer, or may be in the form of a mixture of stereoisomers. Furthermore, it will be appreciated that certain of the compounds disclosed herein contain one or more double bonds and these double bonds can be either Z or E, unless otherwise indicated. In certain embodiments, the compounds of the invention are enantiopure compounds. In certain other embodiments, a mixture of stereoisomers or diastereomers are provided.

Additionally, in another aspect, the present invention provides pharmaceutically acceptable derivatives of the inventive compounds, and methods of treating a subject using these compounds, pharmaceutical compositions thereof, or either of these in combination with one or more additional therapeutic agents. The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety that is susceptible to removal in vivo yielding the parent molecule as the pharmacologically active species. An example of a pro-drug is an ester which is cleaved in vivo to yield a compound of interest. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

Certain compounds of the present invention, and definitions of specific functional groups are also described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups. By the term “protecting group”, has used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, as detailed herein, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM (p-methoxybenzyloxymethyl ether), to name a few), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals. In certain other exemplary embodiments, nitrogen protecting groups are utilized. These nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example of inflammatory disorders, cancer, and other disorders, as described generally above. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (cyclic, acyclic, substituted, unsubstituted, branched or unbranched) having I-6 carbon atoms.

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl-n, hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “alkoxy” (or “alkyloxy”), or “thioalkyl” as used herein refers to an alkyl group, as previously defined, attached to the parent molecular moiety through an oxygen atom or through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. The term “dialkylamino” refers to a group having the structure —N(R′)₂, wherein R′ is alkyl, as defined herein. The term “aminoalkyl” refers to a group having the structure NH₂R′—, wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. It will also be appreciated that aryl and heteroaryl moieties, as defined herein may be attached via an alkyl or heteroalkyl moiety and thus also include -(alkyl)aryl, -(heteroalkyl)aryl, -(heteroalkyl)aryl, and -(heteroalkyl)heteroaryl moieties. Thus, as used herein, the phrases “aryl or heteroaryl” and “aryl, heteroaryl, -(alkyl)aryl, -(heteroalkyl)aryl, -(heteroalkyl)aryl, and (heteroalkyl)heteroaryl” are interchangeable. Substituents include, but are not limited to, any of the previously mentioned substituents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl and the like. In certain embodiements of the present invention, the term “heteroaryl”, as used herein, refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additionally, it will be appreciated, that any two adjacent groups taken together may represent a 4, 5, 6, or 7-membered cyclic, substituted or unsubstituted aliphatic or heteroaliphatic moiety. Additional examples of generally applicable substituents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic or hetercyclic moieties, may optionally be substituted with substituents including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additionally, it will be appreciated that any of the cycloaliphatic or heterocycloaliphatic moieties described above and herein may comprise an aryl or heteroaryl moiety fused thereto. Additional examples of generally applicable substituents are illustrated by the specific embodiments shown in the Examples that are described herein.

The term “heteroaliphatic”, as used herein, refers to aliphatic moieties which contain one or more oxygen sulfur, nitrogen, phosphorus or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additionally, it will be appreciated that any of the cycloaliphatic or heterocycloaliphatic moieties described above and herein may comprise an aryl or heteroaryl moiety fused thereto. Additional examples of generally applicable substituents are illustrated by the specific embodiments shown in the Examples that are described herein.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6- or 7-membered ring or a polycyclic group, including, but not limited to a bi- or tr-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a substituted or unsubstituted aryl or heteroaryl ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one or more of the hydrogen atoms thereon with but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ—OCON(Rₓ)₂; —N(Rₓ)₂; —S(O)₂Rₓ; —NRₓ(CO)Rₓ wherein each occurrence of Rx independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substitutents described above and herein may be substituted or unsubstituted. Additional examples or generally applicable substituents are illustrated by the specific embodiments shown in the Examples which are described herein.

3) Uses, Formulation and Administration

Pharmaceutical Compositions

As discussed above, in one aspect, the present invention provides novel compounds that have biological properties useful for the treatment of disorders resulting from an inappropriate apoptotic response (e.g., excessive or insufficient response). In certain embodiments, the inventive compounds as useful for the treatment of disorders resulting from an insufficient apoptotic response. In certain embodiments of special interest, the compounds of the invention are useful for the treatment of cancer, autoimmune diseases, restenosis, and persistent infections.

Accordingly, in another aspect of the present invention, pharmaceutical compositions are provided, which comprise any one of the compounds described herein (or a prodrug, pharmaceutically acceptable salt or other pharmaceutically acceptable derivative thereof), and optionally comprise a pharmaceutically acceptable carrier. In certain embodiments, these compositions optionally further comprise one or more additional therapeutic agents. Alternatively, a compound of this invention may be administered to a patient in need thereof in combination with the administration of one or more other therapeutic agents. For example, additional therapeutic agents for conjoint administration or inclusion in a pharmaceutical composition with a compound of this invention may be an approved anticancer agent or antiviral or antibacterial agent, or it may be any one of a number of agents undergoing approval in the Food and Drug Administration that ultimately obtain approval for the treatment of any disorder resulting from an inappropriate apoptotic response. It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A. C. S. Symposium Series, and in Edward B. Roche, ed., Bioreversible Carriers in Drug Design, American Pharmaceutical Association and Pergamon Press, 1987, both of which are incorporated herein by reference.

As described above, in certain embodiments, pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Uses of Compounds of the Invention

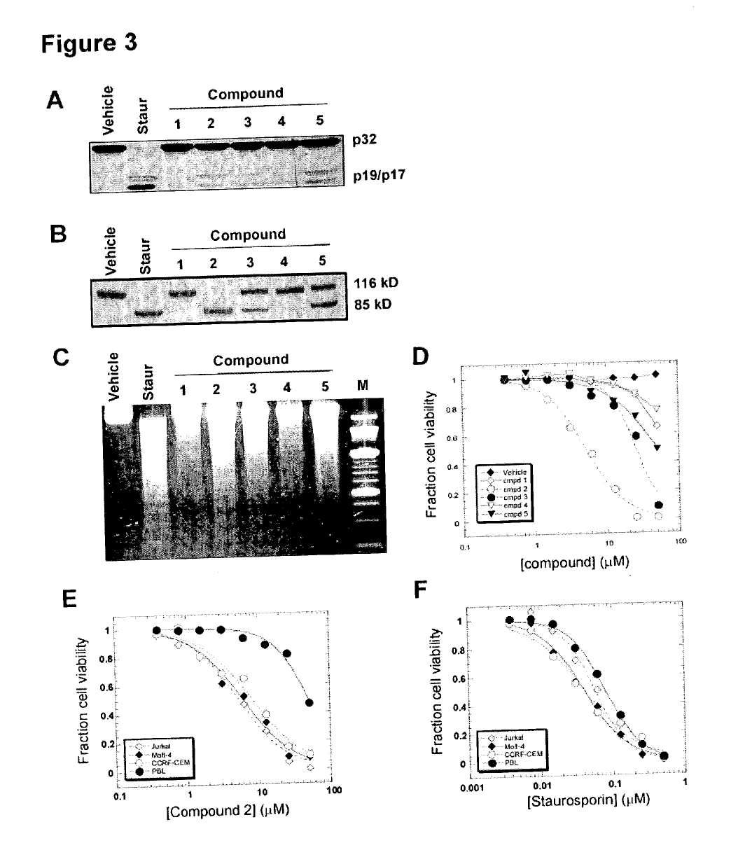

As described in more detail herein, in certain exemplary embodiments, the present invention provides compounds useful for the treatment of disorders resulting from an inappropriate (e.g., excessive or insufficient) apoptotic response. In certain embodiments of special interest, compounds of the invention are useful as activators of apoptosis. As detailed in the exemplification herein in assays to determine the ability of exemplary compounds to induce apoptosis in live cells, certain exemplary compounds exhibited the ability to induce significant processing of procaspase-3 in Jurkat cells. Additionally, the ability of certain compounds to affect cell viability was examined and in certain exemplary embodiments, compounds in the indolone series showed strong cytotoxic activity. In certain embodiments of special interest, compounds exhibit EC₅₀s in the range of approximately 5 μM (see FIG. 3A).

As discussed above, in certain embodiments, compounds of the invention exhibit the ability to induce apoptosis and as such exhibit cytotoxic activity. Thus, compounds of the invention are particularly useful for the treatment of cancer and other disorders resulting from insufficient apoptotic activity.

Thus, as described above, in another aspect of the invention, a method for the treatment of disorders resulting from an inappropriate apoptotic response is provided comprising administering a therapeutically effective amount of a compound of formula (I), as described herein, to a subject in need thereof. It will be appreciated that the compounds and compositions, according to the method of the present invention, may be administered using any amount and any route of administration effective for the treatment of disorders resulting from an inappropriate apoptotic response. For example, in certain exemplary embodiments, compounds of the invention are useful as inducers of apoptosis and thus can be used for the treatment of disorders including, but not limited to, cancer, autoimmune disorders, restenosis, and persistent infections. Thus, the expression “effective amount” as used herein, refers to a sufficient amount of agent to induce apoptosis and thus exhibit a cytotoxic effect. The exact amount required will vary from subject to subject, depending on the species, age, and general condition of the subject, the severity of the infection, the particular therapeutic agent, its mode of administration, and the like. The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, in certain embodiments, after formulation with an appropriate pharmaceutically acceptable carrier in a desired dosage, the pharmaceutical compositions of this invention can be administered to humans and other animals orally, rectally, parenterally, intracistemally, intravaginally, intraperitoneally, topically (as by powders, ointments, or drops), bucally, as an oral or nasal spray, or the like, depending on the severity of the infection being treated. In certain embodiments, the compounds of the invention may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

In certain embodiments, the active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In certain embodiments, the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent for example), or they may achieve different effects (e.g., control of any adverse effects).

### Treatment Kits

In other embodiments, the present invention relates to a kit for conveniently and effectively carrying out the methods in accordance with the present invention. In certain embodiments, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### Equivalents

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### Exemplification

The compounds of this invention and their preparation can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

I) Synthesis of Exemplary Compounds:

As described generally below, a variety of methods can be utilized to synthesize the compounds of the invention.

A. Secondary Amides

In general, compounds represented by the general structures of compounds A-E (and other compounds similar to these compounds) can be synthesized by reaction of 1 equivalent methoxyacetyl chloride with 1 equivalent of the appropriate amine and 3 equivalents of diisopropyl ethylamine in dichloromethane as shown directly below.

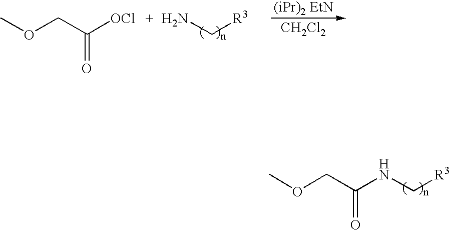

Additionally, it will be appreciated that instead of utilizing methoxyacetyl chloride, ethyl chloroformate can be utilized to generate compounds similar to compound F. For example, compound F was synthesized by reacting 1 equivalent of ethyl chloroformate with 1 equivalent of 2,3-dimethyl-benzyl amine and 3 equivalents of diisopropyl ethylamine in dichloromethane.

Resulting compounds are purified and characterized using standard techniques (e.g., mass spectrometry, NMR).

B. Indalone Series

In general, compounds of the type of compounds M and N can be prepared by reacting a desired indolone with an appropriate bromomethyl reagent (e.g., having a substituted benzene or other aromatic moiety) in the presence of NaH to effect addition of the indolone and displacement of the bromide. For example, compounds M and N was prepared by mixing 0.5 mmol 4-bromomethyl-1,2-dichlorobenzene with 0.5 mmol of the appropriate indolone and 0.6 mmol sodium hydride in 2 mL tetrahydrofuran. The reaction was stirred at 25° C. for 30 min and then extracted with ethyl acetate/water.

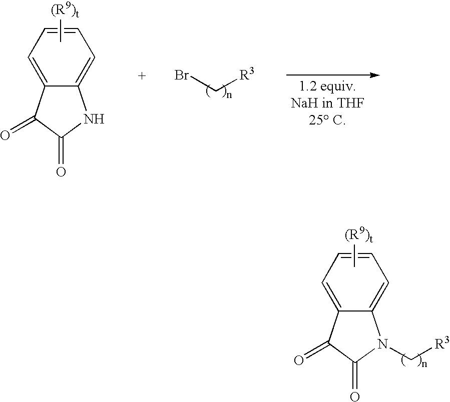

Additionally, compounds of the type of compound O (whereby the carbonyl group is reduced to a methylene group) can be prepared by refluxing compounds of the type of compound L (having both carbonyl moieties) in the presence of hydrazine. For example, compound O was prepared by refluxing 100 mg of compound L in ˜5 mL hydrazine for 2 hr. After HPLC purification, compounds were verified by mass spectrometry.

C. O-Side Carbamates

In general, carbamates as depicted generally below can be prepared by reaction of a desired alcohol with an appropriately substitued isocyanate, as depicted below in the presence of toluene at 80° C. overnight.

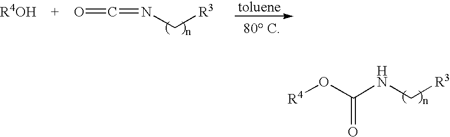

For example, in one embodiment, an appropriate alcohol can be reacted with 0.5 mmol 1,2-dichloro-4-isocyanatomethyl-benzene in 1 mL toluene at 80° C. overnight. After HPLC purification, identity of the compounds were confirmed by mass spectrometry.

D. N-Side Carbamates

In general compounds of the general class depicted below can be prepared from an appropriate carbamate (in certain special embodiments where R⁴ is a lower alkyl group (e.g., methyl, ethyl, propyl, to name a few).

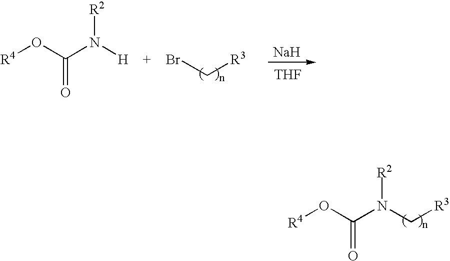

In general, in one exemplary embodiment (where R³ is a disubstituted benzene moiety) 0.5 mmol 4-bromomethyl-1,2-dichlorobenzene was reacted with the appropriate carbamate (0.5 mmol) and 0.6 mmol sodium hydride in 2 mL tetrahydrofuran at 50° C. for 4 hr. After extraction with ethyl acetate/water, compounds were HPLC purified and identity was verified by mass spectrometry.

2) Structures of Exemplary Compounds:

As described above, a variety of exemplary compounds having apoptosis moldulating activity can be synthesized. Detailed below are several exemplary, but non-limiting, examples of compounds of the invention.

| COMPOUND # | STRUCTURE |
|---|---|
| A | 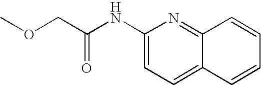 |
| B | 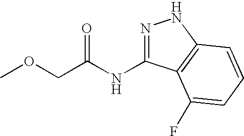 |
| C | 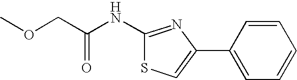 |
| D | 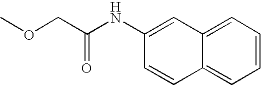 |
| E | 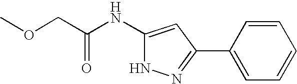 |
| F | 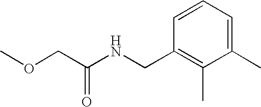 |
| G | 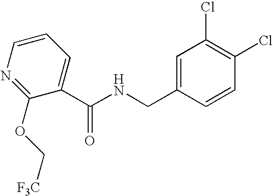 |
| H | 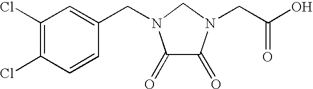 |
| I | 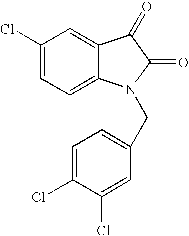 |
| J | 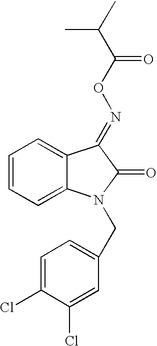 |
| K | 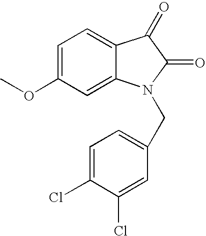 |
| L | 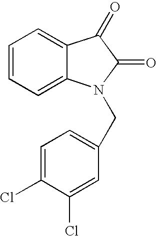 |
| M | 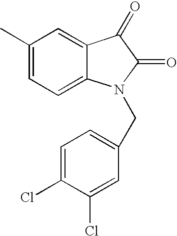 |
| N | 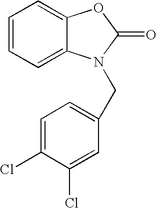 |
| O | 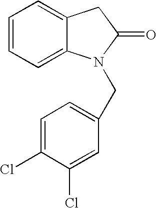 |
| P | 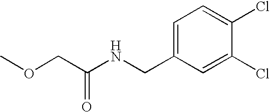 |
| Q | 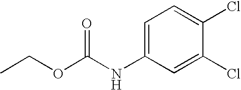 |
| R | 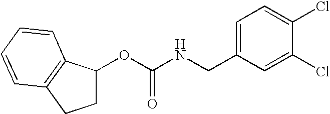 |
| S | 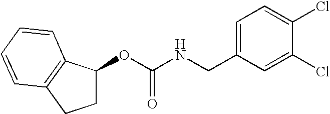 |
| T | 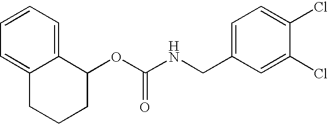 |
| U | 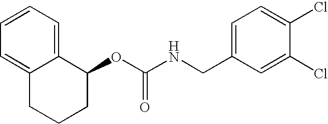 |
| V | 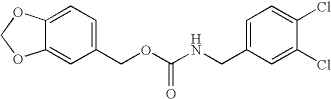 |
| W | 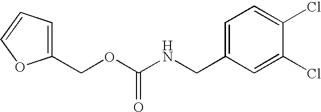 |
| X | 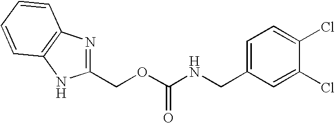 |
| Y | 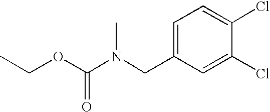 |
| Z | 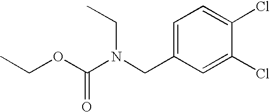 |
| AA | 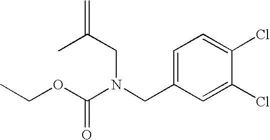 |
| BB | 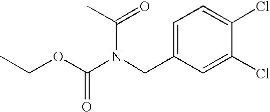 |
| CC | 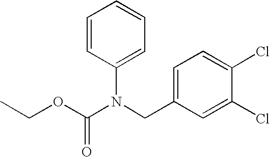 |
| DD | 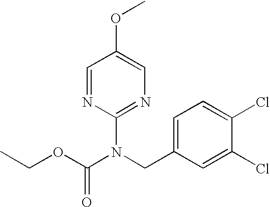 |

3) Biological Data:

In order to identify compounds that modulate apoptosis, a chemical genetics approach was utilized to screen for molecules that target components of the apoptosis pathway (see, T. U. Mayer et al., Science 286, 971-4 (1999)). An in vitro screen using cytoplasmic extracts was adapted for apoptosis to a 96-well format and used to test an in-house library of ˜3500 compounds (see, T. Fernandes-Alnemri et al, Proc Natl Acad Sci USA 93, 7464-9 (1996); X. Liu, C. N. Kim, J. Yang, R. Jemmerson, X. Wang, Cell 86, 147-57 (1996); L. M. Leoni et al., Proc Natl Acad Sci USA 95, 9567-71 (1998)). Briefly, individual compounds were added to HeLa cell S-100 cytoplasmic extracts at a final concentration of 1 mM and apoptosis was induced by adding bovine heart cyto c (HeLa cell cytoplasmic extracts were prepared according to previously published reports (see, P. Li et al., Cell 91, 479-89 (1997)). Compounds were distributed into 96-well microtiter plates at a final concentration of 1 mM. To each well was added 250 μg of total protein from cytoplasmic extracts in HEB buffer (50 mM Hepes pH 7.4, 50 mM KCl, 5 mM EGTA, 2 mM MgCl), with 2 mM DTT, 2 μM cyto c, and 0.5 μM DEVD-AFC substrate in a total of 150 μL. Plates were incubated at 37° C. and fluorescence was read in a LJL Biosystems plate reader at 10 min intervals). Activation of caspase-3 was monitored by cleavage of a fluorogenic substrate, Asp-Glu-Val-Asp-7-amino-4-trifluoromethylcoumarin (DEVDase activity). As designed, the DEVDase fluorescence screen encompasses the release of cyto c from the mitochondria to the activation of caspase-3. Possible targets include cyto c, Apaf-1, caspase-9, caspase-3, inhibitors of apoptosis (IAPs), and possible unidentified components present in the cytosol.

From this screen, 76 compounds were identified that inhibited and 116 compounds that activated DEVDase activity as compared to the DMSO control (FIG. 1A). Representative data for compounds inhibiting and activating DEVDase activity is shown (FIG. 1B). Detailed characterization is presented here of the small molecule apoptosis activators. Because many compounds had intrinsic fluorescence, compounds that activated DEVDase activity were subjected to a secondary screen to directly visualize procaspase-3 processing (FIG. 1C). Upon activation, caspases in the apoptosis cascade are cleaved to give large and small subunits that heterodimerize to form the active protease (see, N. A. Thomberry, Y. Lazebnik, Science 281, 1312-6 (1998)), and this cleavage event can be monitored by immunoblot. Again, lysates were incubated with compound in the presence of cyto c and dATP and probed with an antibody specific for the p 17 large subunit of active caspase-3. Of the 116 activators, 42 caused increased procaspase-3 processing in the secondary screen and were resynthesized as purified compounds. Rescreening of the 42 purified compounds in both assays identified 20 that activated procaspase-3 processing.

Of the 20 validated activators, compound 1 (FIG. 1D) was the most active and was chosen as a lead for the synthesis of more directed chemical libraries. Preliminary exploration into the structure-activity relationship around compound 1 showed that the positions of the two chlorines in the dichlorobenzyl moiety were important for activity. Equally important were the nitrogen and carbonyl groups of the carbamate moiety. Libraries were synthesized that maintained these functional groups, and three additional compounds were identified that strongly activated DEVDase activity (compounds 2, 3 and 5, FIG. 1D). Interestingly, compound 4, the enantiomer of compound 5, had no activity in the in vitro fluorescence assay, strongly suggestive that compound 5 was acting through a specific mechanism.

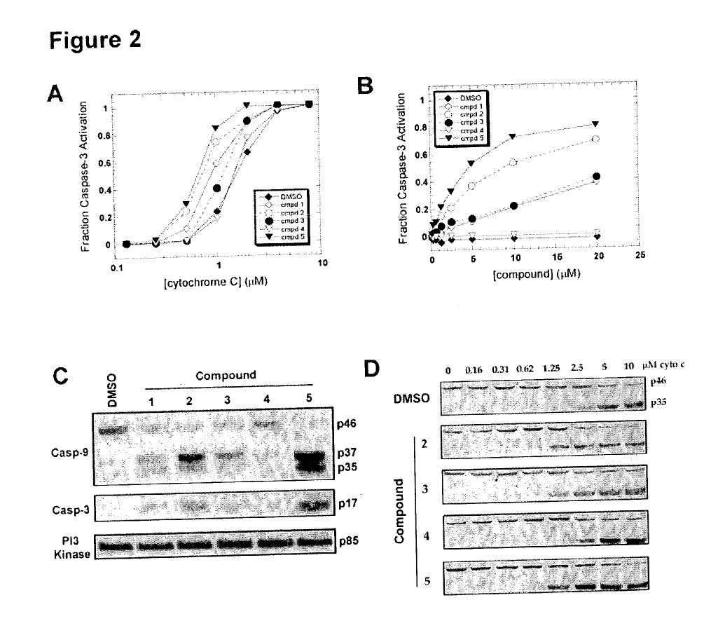

In order to determine if these compounds required cyto c for caspase-3 activation, cyto c was titrated into HeLa cell extract in the presence and absence of compounds and procaspase-3 processing was monitored by capture ELISA (Cyto c was titrated into lysates as detailed in the screening procedure. Capture ELISA assay for active caspase-3 was adapted from Aragones et al., www.biocarta.com. Briefly, a mouse monoclonal antibody recognizing both full-length and cleaved caspase-3 (capture antibody, Transduction Laboratories) was immobilized onto an immunosorp plate and blocked with 5% milk, and 25 μL of the caspase activation reaction in 75 μL Superblock (Pierce) was added. Active caspase-3 was detected by a second rabbit polyclonal antibody that recognizes the cleavage site at D175 (detection antibody, Cell Signaling). Horseradish peroxidase-conjugated goat anti-rabbit secondary antibody and TMB susbtrate were used for detection). As shown in FIG. 2A, titration of cyto c gave a dose-dependent curve for procaspase-3 processing with concentration for half-maximal activation (AC₅₀) at ˜1.75 μM. The addition of 20 μM compounds in most cases resulted in the activation of caspase-3 at a lower concentration of cyto c. However, in all cases the compounds failed to activate at cyto c concentrations below 0.25 μM, suggestive that the compounds act synergistically with cyto c but cannot replace it. Of the compounds tested, compounds 2 and 5 showed the strongest activity, reducing the cyto c AC₅₀ greater than 2-fold to 0.75 μM. The enantiomer of compound 5, compound 4, had no effect. Furthermore, all compounds, with the exception of compound 4, gave a dose-dependent increase in activation when titrated into lysates at a suboptimal concentration of cyto c (FIG. 2B).

To directly visualize the processing of procaspases induced by the compounds, samples at the 20 μM compound concentration in FIG. 2B were assayed by immunoblot for the cleavage of procaspases-9 and -3. As shown in FIG. 2C, the compounds induced the processing of procaspase-9 from the 46 kD inactive form to give a large subunit with bands corresponding to 37 and 35 kD, depending on whether cleavage came from caspase-3 activity or autolytic processing, respectively. Similarly, the compounds induced the processing of procaspase-3 to give the 17 kD large subunit of the active form. The vehicle alone showed no detectable processing of either procaspases, and only limited processing was seen with compound 4.

To better define the step in which the compounds could be acting, the ability of the compounds to affect the processing of procaspase-9 was examined. The activation of caspase-9 occurs prior to the activation of caspase-3, however this process is complicated by the fact caspase-3 will also activate caspase-9 in an amplification loop (see, E. A. Slee et al., J Cell Biol 144, 281-92 (1999)). To examine procaspase-9 processing in the absence of caspase-3, caspase-3 immunodepleted extracts were used and titrated in cyto c in the presence or absence of 20 μM compound (Immunodepletion of caspase-3 from cell extracts was done according to Slee et al., J Biol Chem 276, 7320-6 (2001)). Analysis by immunoblot (FIG. 2D) showed that procaspase-9 was cleaved to give only the 35 kD active form, indicative that all processing came from autolytic cleavage. No activation of caspase-9 was seen at cyto c concentrations below 2.5 μM with the vehicle, whereas with the compound activators processing occurred between 0.62 to 1.25 μM cyto c. Thus, the compounds appear to act prior to caspase-3 activation, possibly by directly activating caspase-9 or by involvement in the formation of the apoptosome complex.

Compounds were then tested for their ability to induce apoptosis in live cells. The ability of the compounds to activate caspase-3 in a Jurkat cell line was first assayed. Cells were incubated with the DMSO vehicle, staurosporin (a potent apoptosis inducer), or compounds for 6 hr and then lysed. Samples were examined by immunoblot for the processing of procaspase-3 from the 32 kD inactive form to the 17 kD active form. As shown in FIG. 3A, staurosporin and compounds 2, 3 and 5 induced significant processing of procaspase-3 in Jurkat cells, whereas less processing was seen with compound 4 (the inactive enantiomer), and no processing was seen with either the vehicle or compound 1 (the parental compound). This same pattern was seen when we assayed for the cleavage of poly (ADP-ribose) polymerase (PARP), a cellular substrate of caspases-3 and -7 (see, D. W. Nicholson, Cell Death Differ 6, 1028-42 (1999)). Upon apoptosis, PAR-P is cleaved from a 116 kD to an 85 kD protein. Staurosporin and compound 2 induced complete cleavage of PARP as judged by the disappearance of the 116 kD band, and compounds 3 and 5 induced limited processing (FIG. 3B).

One of the hallmarks of apoptosis is the fragmentation of chromosomal DNA into discrete, nucleosomal sized bands. The staurosporin-induced fragmentation of Jurkat cell DNA can be visualized as a “laddering” effect when examined by agarose gel (FIG. 3C). Compounds 2, 3 and 5 induced DNA fragmentation in a similar manner as staurosporin, whereas compounds 1 and 4 had a less pronounced effect, and no fragmentation was seen with the vehicle alone. We then examined the effects of the compounds on cell viability. Jurkat cells were incubated at different concentrations of compounds for 22 hr and then assayed by MTT test. Of the compounds tested, the indolone series showed the strongest cytotoxic activity, with compound 2 killing cells with an EC₅₀ of ˜5 μM (FIG. 3D).

Since compound 2 had the most potent cellular activity when tested against Jurkats, it was then tested against a panel of normal and transformed cell lines. Normal cell lines include peripheral blood lymphocytes (PBL) isolated from donated human blood, non-transformed mammary fibroblasts (MCF-10A), human mammary epithelial cells (HMEC), human umbilical vein endothelial cells (HUVEC), and human prostate epithelial cells (PREC). Transformed cell lines include cell lines from leukemia, breast, lung, ovarian, and epidermal cancers (Table 1). In general, normal cell lines were resistant to compound 2 induced apoptosis. Of the normal cell lines tested, compound 2 had an IC₅₀ of 50 μM when tested against PBLs, and ˜43 nM when tested against HUVECs, but had no effect except at the highest concentration (50 μM) when tested against MCF 10A cells, HMEC cells, or PREC cells (Table 1). On the other hand, when tested against the three leukemia cell lines in the panel, compound 2 induced killing with IC₅₀s ranging from 4 to 9 μM, potentially giving a 5- to 10-fold therapeutic window when compared to the IC₅₀ for PBLs (FIG. 3E). Significantly, all four lymphocyte cell lines, normal and cancerous, were equally susceptible to staurosporin-induced cytotoxicity (FIG. 3F).

TABLE 1

| | | p53 | Staurosporin | Compound 2 |
|---|---|---|---|---|
| Cell type | Cell name | status | IC₅₀ (μM) | IC₅₀ (μM) |
| Normal Cell | | | | |
| | PBL | + | 0.069 | 50 |
| | MCF 10A | + | 0.126 | >50 |
| | HMEC | 0.109 | >50 | |
| | HUVEC | + | 0.063 | 43 |
| | PREC | + | 0.364 | >50 |
| Cancer | | | | |
| Cell Line | | | | |
| Leukemia | Jurkhat | + | 0.059 | 4 |
| | Molt-4 | + | 0.054 | 6 |
| | CCRF-CEM | − | 0.069 | 9 |
| Breast | BT-549 | − | 0.220 | 20 |
| | MDA-MB-453 | + | >1 | >50 |
| | MDA-MB-468 | − | 0.357 | 44 |
| | MCF-7* | + | >1 | >50 |
| Lung | NCI-H23 | − | 0.085 | 35 |
| Ovarian | SK-OV-3^{#} | − | 0.198 | >50 |
| Epidermal | A431 | 0.059 | 40 | |

*Caspase-3 deficient
^{#}Apaf-1 deficient

Breast, lung and epidermal cancer cell lines had variable sensitivity to compound 2 (Table 1), and this sensitivity did not correlate with p53 status. The fact that p53 status was not a factor in compound 2 mediated killing is consistent with a mechanism of action that centers at the formation of the apoptosome, an event downstream of p53 signaling. In addition, compound 2 has no effect against cell lines that are defective in the caspase-9 pathway the ovarian cancer cell line SK-OV-3, which is deficient in Apaf-1 activity (J. R. Liu et al., Cancer Res 62, 924-31 (2002); B. B. Wolf et al., J Biol Chem 276, 34244-51. (2001)); and the breast cancer cell line MCF-7, which is deficient in caspase-3 activity (R. U. Janicke, M. L. Sprengart, M. R. Wati, A. G. Porter, J Biol Chem 273, 9357-60. (1998))—consistent with biochemical data that suggest that compound 2 acts by promoting the formation of the apoptosome.

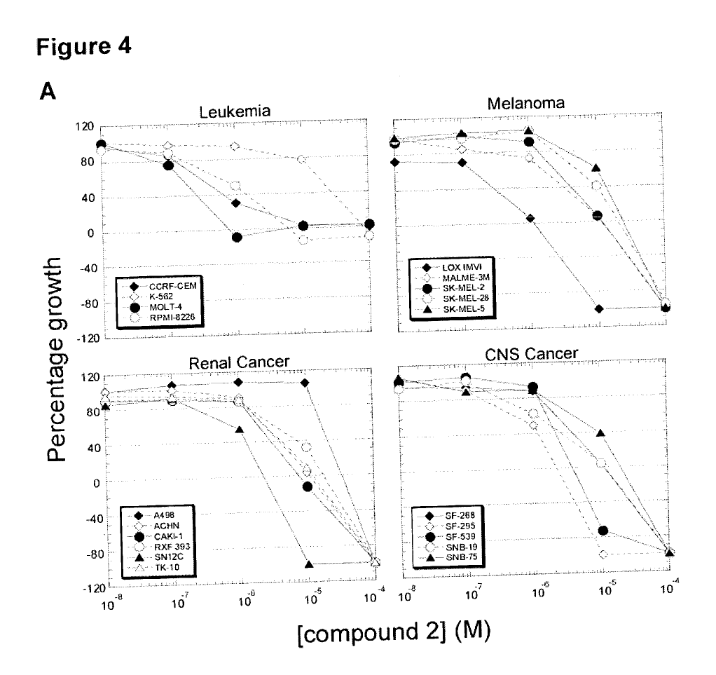

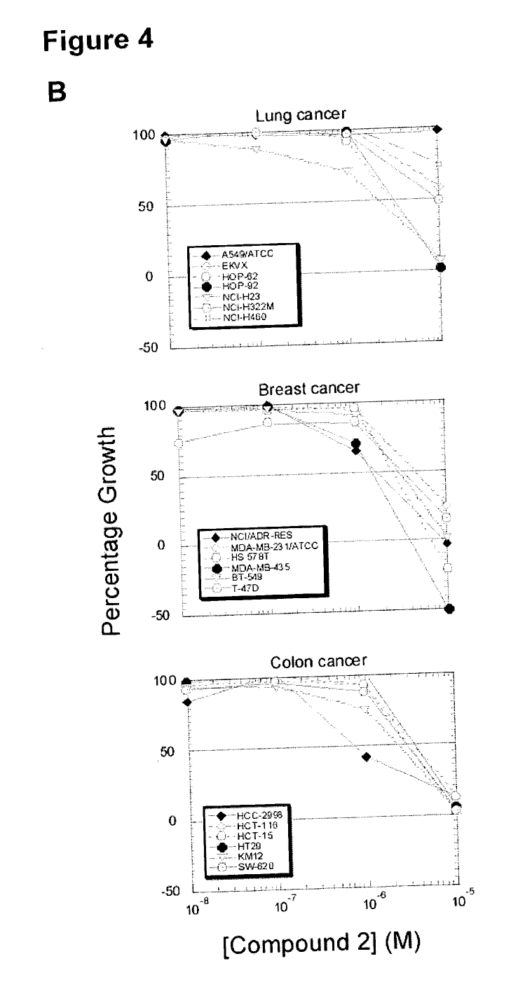

Compounds 2-5 additionally were submitted to the National Cancer Institute for screening in the cancer panel. Consistent with data generated in-house, compound 2 was the most active overall. Compound 2 exerted a cytostatic effect on the majority of cell lines tested, inhibiting cell growth by 50-100% at 10 μM in 40 out of 48 cell lines tested. In addition, compound 2 exerted a cytotoxic effect, reducing the cell numbers by 10-50% in four cell lines and by 50-100% in eight cell lines from the initial levels when tested at 10 μM. At 100 μM compound 2 exhibited 100% cytotoxicity in virtually all cell lines, which may be due to nonspecific effects. A subset of cell lines were particularly sensitive to the effects of compound 2, exhibiting 5-10 fold greater sensitivity than the mean response of the panel; these include the lymphoid cell lines CCRF-CEM and MOLT-4, the melanoma cell line LOX IMVI, the renal cancer cell line SN 12C, and the CNS cancer cell line SF-295 (FIG. 4A). Representative dose-response plots for lung, breast and colon cancer cell lines are also shown (FIG. 4B). Results for ovarian and prostate cancer were similar.

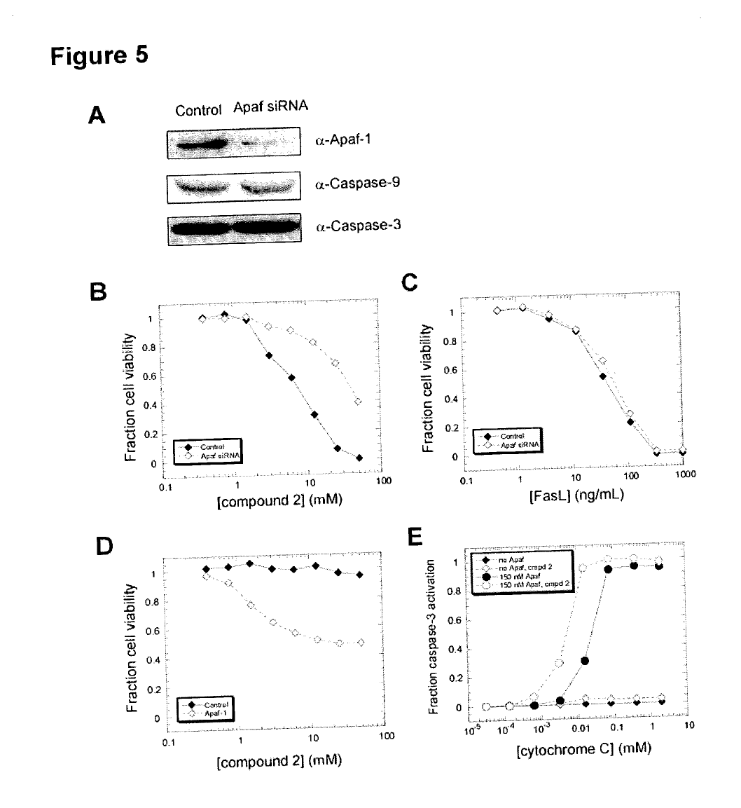

In order to verify that compound 2 targets the apoptosome as a function of its cytotoxic activity, small interfering RNA (siRNA) was used to silence the expression of Apaf-1 in Jurkat cells. Sense and anti-sense oligonucleotides corresponding to nucleotides 978-998 of Apaf-1 ((AATTGGTGCACTTTTACGTGA) as reported by Lassus et al. Science 297, 1352-4. (2002)), were purchased from Dharmacon. Transfection of siRNA into Jurkat cells was accomplished using GeneSilencer (Gene Therapy Systems) according to manufacturer's instructions. There was a significant decrease in the level of expression of Apaf-1 48 hr after transfection of an Apaf-1 specific siRNA, without affecting the expression of caspases in the pathway (FIG. 5A). Reducing the expression of Apaf-1 resulted in resistance of Jurkat cells to compound 2 induced cell killing (FIG. 5B), providing strong evidence that Apaf-1 is required for the activity of compound 2 in cells. This resistance does not result from a non-specific protective mechanism of the Apaf-1 siRNA as these cells are still sensitive to apoptosis induced by Fas Ligand, which proceeds through the caspase-8 pathway (FIG. 5C).

If silencing Apaf-1 in Jurkat cells make them resistant to compound 2 induced apoptosis, then it would be expected that ectopic expression of wildtype Apaf-1 in an Apaf-1 deficient cell line should render cells sensitive to compound 2. As mentioned above, the ovarian cancer cell line SK-OV-3 is deficient in Apaf-1 activity and is resistant to compound 2. Apaf-i was cloned into pcDNA-3 and transiently transfected into SK-OV-3 cells using Fugene (Roche) according to the manufacturer's instructions. Transient transfection of Apaf-1 into SK-OV-3 cells rendered them sensitive to compound 2 cytotoxicity as compared to the vector control (FIG. 5D). In several independent experiments, the viability of Apaf-1 transfected SK-OV-3 cells exposed to compound 2 leveled between 40-50%, perhaps reflecting the transfection efficiency. The requirement for Apaf-1 for compound 2 activity was also shown in experiments involving cell lysates from untransfected SK-OV-3 cells. As shown in FIG. 5E, lysates from SK-OV-3 cells do not show normal processing of procaspase-3 in response to dATP and cyto c, either in the presence or absence of compound 2. However, the addition of purified Apaf-1 to the lysates resulted in very strong processing of procaspase-3 in response to cyto c in a dose-dependent manner. The addition of 20 μM compound 2 shifted the activation curve to the left, such that activation occurred at a lower concentration of cyto c as seen with the HeLa cell lysates. These data clearly show that Apaf-1 is required for the activity of compound 2, both in biochemical assays and in whole cells.

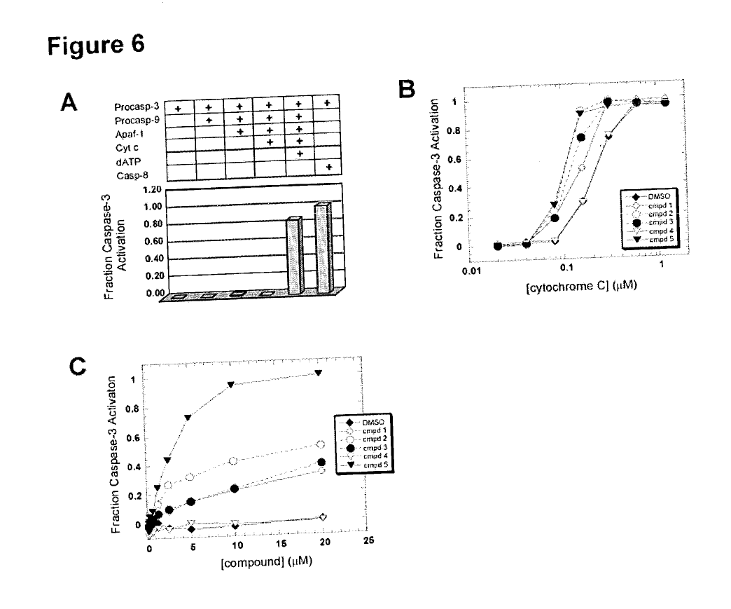

Next, we wished to determine whether one of the proteins of the purified system was a target of the compounds. Previous data demonstrate that caspase-3 activation can be reconstituted using only purified proteins (see, H. Zou, Y. Li, X. Liu, X. Wang, J Biol Chem 274, 11549-56 (1999)). In the presence of dATP, cyto c, Apaf-1 and procaspase-9 will oligomerize, resulting in the cleavage and activation of procaspase-9 (see, A. Saleh, S. M. Srinivasula, S. Acharya, R. Fishel, E. S. Alnemri, J Biol Chem 274, 17941-5 (1999)). Active caspase-9 then cleaves and activates procaspase-3. To determine the purified protein target of the compounds, we cloned and expressed human Apaf-1 (Apaf-1 XL, see, Y. Hu, M. A. Benedict, L. Ding, G. Nunez, EMBO J. 18, 3586-95 (1999)) and procaspase-9; cyto c and procaspase-3 were purchased from commercial vendors. (The XL form of Apaf-1 was cloned into pFastbacHT and expressed in Hi-5 cells. Procaspase-9 was cloned into pDest17 and expressed in E. coli. Purification of Apaf-1 and procaspase-9 were according to the following procedures, respectively: P. Li et al., Cell 91, 479-89 (1997) and H. Zou, Y. Li, X. Liu, X. Wang, J Biol Chem 274, 11549-56 (1999)). Procaspase-3 was purchased from Biomol, and bovine heart cyto c was purchased from Sigma). Addition of all four components in the presence of dATP resulted in the efficient cleavage of procaspase-3 (FIG. 6A), and the titration of cyto c gave a dose-dependent curve for the activation of caspase-3 similar to what was seen with cell extracts (FIG. 6B). The addition of 20 μM compounds to the reaction caused the processing of procaspase-3 to occur at a reduced cyto c concentration (FIG. 6B). In addition, we obtained dose response curves for the compound-induced activation by titrating compounds at a cyto c concentration that produced 25% activation (0.15 μM in the purified system, FIG. 6C). These data clearly show that the compounds act upon one or more of the components of the purified system. The addition of compounds to either procaspase-9 or procaspase-3 alone did not induce the activation of either proteins, suggestive that the compounds required cyto c, Apaf-1, or both for their activity.

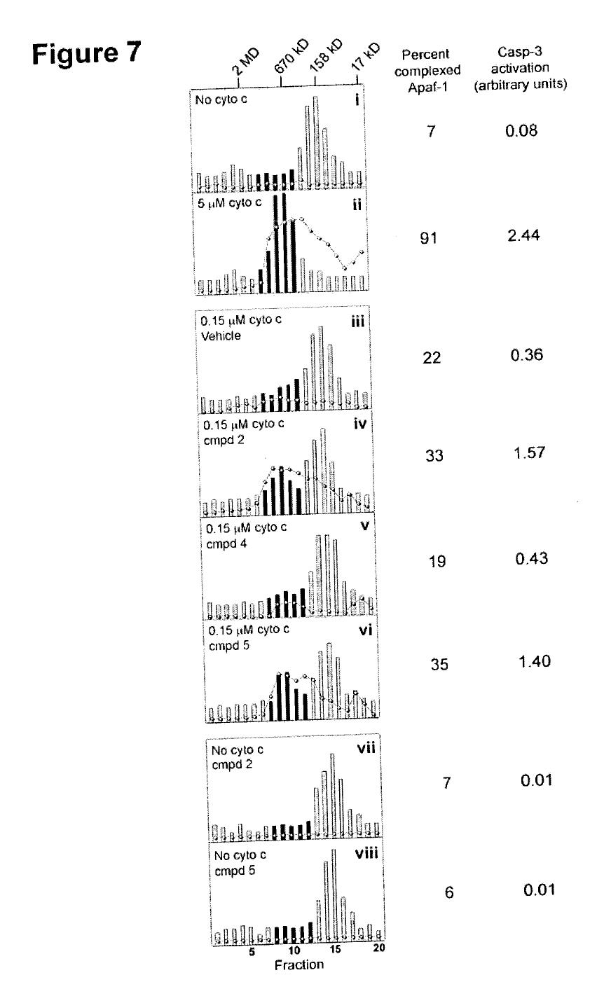

As the compounds appeared to be acting prior to caspase activation, their effect on the oligomerization of Apaf-1 was assessed (K. Cain, D. G. Brown, C. Langlais, G. M. Cohen, J Biol Chem 274, 22686-92 (1999); K. Cain et al., J Biol Chem 275, 6067-70 (2000)). By gel filtration, Apaf-1 normally runs as a monomer of ˜140 kD (FIG. 7) (see, A. Saleh, S. M. Srinivasula, S. Acharya, R. Fishel, E. S. Alnemri, J Biol Chem 274, 17941-5 (1999)). Incubation of Apaf-1 with 5 μM cyto c induces over 90% of the Apaf-1 to form large complexes of ˜700 kD, whereas incubation with 0.15 μM cyto c concentration allowed only 22% of the Apaf-1 to assemble into complexes. However, the addition of 20 μM compound 2 or 5 at the lower cyto c concentration caused the formation of two higher molecular weight complexes: a major peak that runs at the same retention time as the complex seen at 5 μM cyto c (˜700 kD), and a minor peak that elutes in the void volume. Compound 4 had no effect on Apaf-1 oligomerization in contrast to what was seen with the active enantiomer, compound 5. Significantly, the addition of compounds 2 or 5 to Apaf-1 in the absence of cyto c resulted in no complex assembly. To determine if the Apaf-1 complexes induced by the compounds at the lower cyto c concentration are competent for caspase activation, procaspase-9, procaspase-3, and dATP were added to fractions from the gel filtration column and assayed for caspase-3 activation. Fractions from samples that had been incubated with 5 μM cyto c strongly activated caspase-3 processing (FIG. 7, panel ii, line graph), with the maximal caspase activation corresponding to the fractions that contained the apoptosome complex. On the other hand, no fractions form the sample without cyto c activated capsase-3 processing (panel i). There was a small but measurable amount of capsase-3 activation when the cyto c concentration was reduced to 0.15 μM (panel iii). However, the extent of caspase-3 activation at the reduced level of cyto c increased approximately 4-fold when either compound 2 or 5 was added to the reaction (from 0.36 arbitrary units for the vehicle alone to 1.57 and 1.4 for compounds 2 and 5, respectively, panels iv and vi). Thus, while compound 2 and 5 increased the oligomerization of Apaf-1 about 1.5-fold over the vehicle alone, this effect resulted in an approximately 4-fold increase in caspase-3 activation. These data are consistent with an amplification cascade whereby a modest effect upstream (in terms of Apaf-1 oligomerization) is magnified at later stages (in terms of caspase-3 activation). Again, there was no increase in capsapse-3 activation when compounds were incubated with Apaf-1 in the absence of cyto c.

The identification of small molecule apoptosis activators that function downstream of Bcl-2 with an entirely novel mechanism of action have been reported herein. The two series of activators presented in this study—the indolones (compounds 2 and 3) and the carbamates (compounds 4 and 5)—function to promote Apaf-1 oligmerization in a cyto c-dependent manner; the indolones are more potent as cytotoxic agents whereas the carbamates show strong stereospecificity. To our knowledge, this report is the first description of compounds that induce the formation of the apoptosome, placing these compounds among the rare class of small molecules that promote protein-protein interactions.

The fact that these compounds do not bypass the requirement for cyto c and yet are able to induce apoptosis in whole cells suggests that low but unproductive levels of cyto c may be present in the cytosol. Many types of cancers have elevated levels of Bcl-2 or reduced levels of pro-apoptotic Bax, in both cases resulting in decreased cyto c translocation (see, G. Kroemer, J. C. Reed, Nat Med 6, 513-9 (2000)). Cell-permeable small molecules that will promote the activation of caspases at reduced levels of cytosolic cyto c could play an important role in overcoming chemoresistance, particularly since these apoptosis activators appear to be selective for some types of cancer cells over normal cells. The compounds could also potentially be used in combination with other therapeutics even in cancers having Apaf-1 expression defects. For example, the compounds could potentially be used with a DNA methyltransferase inhibitors in cancers epigenetically silenced for expression of wild-type Apaf-1, such as occurs, e.g., in certain melanomas (M. S. Soengas et al., Nature 409 207-11 (2001)). This study highlights the utility of screening compounds against a signaling pathway, as these compounds could not have been identified by traditional screening against any individual component in the apoptosis cascade.

### EXAMPLES

The following examples are methods that can be used to identify compounds that are apoptosis inducers. Each method involves measurement of the ability of the compounds to increase a protein-protein interaction that is essential for apoptosome formation, i.e., the oligomerization of Apaf-1 and cyto c in the presence of a hydrolyzable nucleoside phosphate such as dATP. Alternatively, certain nonhydrolyzable nucleoside phosphates, such as ADPCP (β,γ-methylene adenosine 5′-triphosphate) may be substituted for the hydrolyzable nucleoside phosphate, as binding of the nucleoside phosphate is possibly more important than the hydrolysis of the nucleoside phosphate (X. Jiang, et al., JBC 275 31199-203 (2000)).

The methods involve: a) combining in a first mixture at least these three components each in a first amount sufficient to promote the oligomerization of at least Apaf-1 and cyto c; b) measuring a first extent of oligomerization; c) combining in a second mixture the same compoments as in the first mixture plus a test compound; d) measuring a second extent of oligomerization; and e) comparing the first extent of oligomerization with the second extent of oligomerization to determine whether the test compound is a modulator of apoptosis.

In description of specific embodiments, the methods are tailored to identify activators of apoptosis by reducing the amount of one of Apaf-1, cyto c, or hydrolyzable nucleoside phosphate. In preferred embodiments, cyto c is present in a reduced amount in the second mixture.

The extent of oligomerization is measured using a variety of known methods. In one embodiment, the extent of oligomerization is measured by quantitating protein-protein binding or by the proportion of either Apaf-1 or cyto c that is present in oligomers or in large particles. In another embodiment, the extent of oligomerization is measured by monitoring the Apaf-1/Apaf-1 interaction. In another embodiment, the extent of oligomerization is measured by monitoring the Apaf-1/cyto c interaction. In another embodiment, the extent of oligomerization is measured by monitoring one of the downstream events from apoptosome formation in the apoptosis pathway. For example, the extent of oligomerization can be measured by monitoring the processing of procaspase-9 to active caspase-9; the activity of caspase-9; the processing of procaspase-3 to active caspase-3; the activity of caspase-3; or even apoptosis itself (if in a cellular assay).

In view of the teachings herein, variations of the specifically described methods will be readily apparent to the skilled artisan. Consequently, the following examples are for illustrative purposes and are not intended to be limiting in any way.

### Example 1

Detection of Apaf-1 Oligomerization by Gel Filtration Chromatography and Caspase-3 Activation

Bovine heart cyto c is purchased from Sigma and used without further purification. The XL form of Apaf-1 is cloned into pFastbacH and expressed in insect cells, resulting in full-length Apaf-XL with a C-terminal six histidine tag. Purification of Apaf-1 is according to A. Saleh, et al., J Biol Chem 274, 17941-45 (1999). 100 μL of 2 μM Apaf-1 is combined with 300 μM dATP in a mixture, with or without the further addition of 0.15 μM cyto c or compounds and heated for 30 min at 37° C.

The mixtures are next injected into a Superose 6 gel filtration column and separated at 0.4 mL/min in PBS; 0.8 mL fractions are collected 10 min after injection. To determine the concentration of Apaf-1 in each column fraction, a 100 μL aliquot is taken from each fraction, to which is added beta-mercaptoethanol to a concentration of 5 mM and Tween-20 to a final concentration of 0.05%. These aliquots are heated to 95° C. for 5 min and used for an Apaf-1 capture ELISA assay. The capture antibody for Apaf-1 is a mouse monoclonal antibody (Transduction Laboratories), and the detection antibody is a rabbit polyclonal (Alexis) antibody; each recognizing different epitopes. The signal for each sample is normalized to the signal for 10 μg Apaf-1. The quantity of the Apaf-1 present in protein complexes of approximately 700 kD is compared with monomeric Apaf, which runs at approximately 140 kD. An increase in the quantity of Apaf-I present in the 700 kD complexes by the compound indicates that the compound is an apoptosis inducer.

For the caspase-3 activation assays, a 75 μL aliquot from each fraction is added to 25 μL S-100 cellular extracts (125 μg total protein), along with DTT, which is added to 2 mM final concentration. Reactions are incubated at 37° C. for 1 hr and then used for caspase-3 capture ELISA. Activation is normalized to the signal for activation at 10 μM cyto c. An increase in the processing of caspase-3 due to compound-enhanced Apaf-1 oligomerization indicates that the compound is an apoptosis inducer.

### Example 2

Detection of Apaf-1 Oligomerization by Fluorometric Microvolume Assay Technology (FMAT)

Monitoring Apaf-1/Apaf-1 Interactions

Apaf-1 is expressed in SF9 insect cells and purified by his-tag affinity chromatography. The protein is then labeled with the NHS-ester of biotin by mixing protein with four molar equivalents of biotin-LC-NHS (Pierce) in 50 mM Na₂CO₃ buffer at pH 9. The protein solution is incubated for 1 hr at room temperature and purified by size-exclusion chromatography (Nap5, Pierce) into phosphate-buffered saline (PBS)+1 mM DTT.

A separate pool of the unlabelled, purified protein is separately labeled with Cy5 dye by mixing Apaf-1 with 4 molar equivalents of Cy5-NHS ester (Amersham) in 50 mM Na₂CO₃ buffer at pH 9. This Apaf-1 solution is incubated for 1 hr at room temperature and purified by size-exclusion chromatography (Nap5, Pierce) into SuperBlock/PBS+1 mM DTT (Pierce).

Biotinylated-Apaf-1 is bound to streptavidin-coated FMAT beads (PE Biosystems) by adding 200 nM protein to 100 μL of beads per 96-well plate in a total of 500 μL PBS. The beads are incubated with the Apaf-1 for 20 min at room temperature, centrifuged, decanted and then resuspended in 1 mL of SuperBlock/PBS+6% glycerol+1 mM DTT.

To optimize the concentrations of Apaf-1-coated beads, Cy5-labeled Apaf-1, and unlabeled cyto c (Sigma), a three-dimensional matrix is run wherein the concentrations of all three components are varied. Briefly, the Apaf-1-bound beads are serially diluted in one direction, and 10 μL of each dilution is added to each well. Next, Cy5-labeled Apaf-1 is serially diluted in a second direction, and 90 μL of each dilution is added to each well. A dilution of unlabeled cyto c (1 μL) is then added to all the wells of each plate. Thus, each well in a plate should contain varying concentrations of beads and Apaf-1, but the same concentration of unlabeled cyto c. Several plates are then used to test different concentrations of unlabeled cyto c. Finally, 2′-deoxyadenosine 5′-triphosphate (dATP) is added to each well to a final concentration of 300 μM. The mixtures in the wells are incubated for 20 min and fluorescence is detected as described below.

Additionally, the compounds to be tested are suspended in DMSO to a final concentration of 100 mM. The compounds are then serially diluted in DMSO and 1.2 μL of each dilution is then transferred to a clean, 96-well plate. These DMSO dilutions are mixed with 120 μL of a protein solution containing the appropriate concentrations of Cy5-labeled Apaf-1 and unlabeled cyto c, as determined above. Ninety microliters of the resulting solution is then transferred to a black, opaque 96-well plate containing 10 μL of Apaf-1-coated beads at the appropriate dilution also as determined above. dATP is added to a final concentration of 300 μM and the mixtures are incubated for 20 min.

Fluorescence is read in an FMAT 8100 HTS System from PE Biosystems by excitation at 633 nm and emission at 690 nm. The oligomerization of Cy5-labeled Apaf-1 onto the Apaf-1-bound FMAT beads is detected by an increase in the percentage of fluorescence seen as large particles. An increase in this percentage in the presence of the compound indicates that the compound is an apoptosis inducer.

Monitoring Apaf-1/Cyto C Interactions

Bovine heart cyto c is purchased from Sigma. The protein is then labeled with the NHS-ester of biotin by mixing protein with four molar equivalents of biotin-LC-NHS (Pierce) in 50 mM Na₂CO₃ buffer at pH 9. The protein solution is incubated for 1 hr at room temperature and purified by size-exclusion chromatography (Nap5, Pierce) into phosphate-buffered saline (PBS)+1 mM DTT.

Apaf-1 is expressed in SF9 insect cells and purified by his-tag affinity chromatography. Protein is then labeled with Cy5 dye by mixing Apaf-1 with 4 molar equivalents of Cy5-NHS ester (Amersham) in 50 mM Na₂CO₃ buffer at pH 9. This Apaf-i solution is incubated for 1 hr at room temperature and purified by size-exclusion chromatography (Nap 5, Pierce) into SuperBlock/PBS (Pierce)+1 mM DTT.

Biotinylated-cyto c is bound to streptavidin-coated FMAT beads (PE Biosystems) by adding 100 nM protein to 100 μL of beads per 96-well plate in a total of 500 μL PBS+1 mM DTT. The beads are incubated with the cyto c for 20 min at room temperature, centrifuged, decanted and then resuspended in 1 mL of SuperBlock/PBS+6% glycerol+1 mM DTT.

To optimize the concentrations of cyto c-coated beads, Cy5-labeled Apaf-1, and unlabeled cyto c, a three-dimensional matrix is run wherein the concentrations of all three components are varied. Briefly, the cyto c-bound beads are serially diluted in one direction, and 10 μL of each dilution is added to each well. Next, Cy5-labeled Apaf-1 is serially diluted in a second direction, and 90 μL of each dilution is added to each well. 1 μL of a dilution of unlabeled cyto c is then added to all the wells of each plate. Thus, each well in a plate should contain varying concentrations of beads and Apaf-1, but the same concentration of unlabeled cyto c. Several plates are then used to test different concentrations of unlabeled cyto c. Finally, 2′-deoxyadenosine 5′-triphosphate (dATP) is added to each well to a final concentration of 300 μM. The mixtures in the wells are incubated for 20 min and fluorescence is detected as described below.

Additionally, the compounds to be tested are suspended in DMSO to a final concentration of 100 mM. The compounds are then serially diluted in DMSO and 1.2 PL of each dilution is then transferred to a clean, 96-well plate. These DMSO dilutions are mixed with 120 μL of a protein solution containing the appropriate concentrations of Cy5-labeled Apaf-1 and unlabeled cyto c, as determined above. Ninety microliters of the resulting solution is then transferred to a black, opaque 96-well plate containing 10 μL of cyto c-coated beads at the appropriate dilution also as determined above. dATP is added to a final concentration of 300 μM and the mixtures are incubated for 20 minutes.

Fluorescence is read in an FMAT 8100 HTS System from PE Biosystems by excitation at 633 m and emission at 690 nm. The oligomerization of Cy5-labeled Apaf-1 onto the cyto c-bound FMAT beads is detected by an increase in the percentage of fluorescence seen as large particles. An increase in this percentage in the presence of the compound indicates that the compound is an apoptosis inducer.

### Example 3

Detection of Apaf-1 Oligomerization by Scintillation Proximity Assay (SPA)

Monitoring Apaf-1/Apaf-1 Interactions

Apaf-1 is expressed in SF9 insect cells and purified by his-tag affinity chromatography. A first pool of the purified, unlabeled protein is labeled with the NHS-ester of biotin by mixing protein with four molar equivalents of biotin-LC-NHS (Pierce) in 50 mM Na₂CO₃ buffer at pH 9. The protein solution is incubated for 1 hr at room temperature and purified by size-exclusion chromatography (Nap5, Pierce) into phosphate-buffered saline (PBS)+1 mM DTT.

Another pool of the unlabeled, purified Apaf-1 is separately labeled with tritiated (³H)-propionic NHS ester (Amersham) by mixing 10 mmol Apaf-1 with 1 mCi propionic acid in 50 mM Na₂CO₃ buffer at pH 9. The resulting solution is incubated for 1 hr at room temperature and purified by size-exclusion chromatography (Nap 5, Pierce) into PBS (Pierce)+1 mM DTT.

Biotinylated Apaf-1 is bound to streptavidin-coated scintillation beads (Amersham) by adding 200 nM protein to 10 mL of beads per 96-well plate. The beads are incubated for 20 min at room temperature, centrifuged, decanted, and then resuspended in 1 mL of SuperBlock/PBS+6% glycerol+1 mM DTT.

To optimize the concentrations of Apaf-1-coated beads, ³H-labeled Apaf-1, and unlabeled cyto c (Sigma), a three-dimensional matrix is run wherein the concentrations of all three components are varied. Briefly, the Apaf-1-bound beads are serially diluted in one direction, and 10 μL of each dilution is added to each well. Next, ³H-labeled Apaf-1 is serially diluted in a second direction, and 90 μL of each dilution is added to each well. Then 1 μL of a dilution of unlabeled cyto c is added to all the wells of each plate. Thus, the wells in a single plate should contain varying concentrations of beads and Apaf-1, but the same concentration of the unlabeled cyto c. Several plates are then used to test different concentrations of the unlabeled cyto c. Finally, 2′-deoxyadenosine 5′-triphosphate (dATP) is added to each well to a final concentration of 300 μM. The mixtures in the wells are incubated for 20 min and scintillation is detected as described below.

Compounds to be tested are suspended in DMSO to a final concentration of 100 mM. The compounds are then serially diluted by three-fold dilutions in DMSO and 1.2 μL of each dilution is then transferred to a clean, 96-well plate. The DMSO solutions are mixed with 120 μL of a solution containing the appropriate concentrations of ³H-labeled Apaf-1 and unlabeled cyto c as determined above. Ninety microliters of the resulting solution is then transferred to the clear bottom 96-well plate containing 10 μL of Apaf-1-coated beads at the appropriate dilution also as determined above. dATP is added to a final concentration of 300 μM and the resulting mixture is incubated for 20 min.

Scintillation is read in a Wallac Microbeta Scintillation Counter. Scintillation arises from binding of ³H-labeled Apaf-1 to the scintillant-containing beads; increase in the scintillation is due to induction of the protein-protein interaction by the compounds.

Monitoring Apaf-1/Cyto C Interactions

Bovine heart cyto c is purchased from Sigma. The protein is then labeled with the NHS-ester of biotin by mixing protein with four molar equivalents of biotin-LC-NHS (Pierce) in 50 mM Na₂CO₃ buffer at pH 9. The protein solution is incubated for 1 hr at room temperature and purified by size-exclusion chromatography (Nap5, Pierce) into phosphate-buffered saline (PBS)+1 mM DTT.

Apaf-1 is expressed in SF9 insect cells and purified by his-tag affinity chromatography. Protein is then labeled with tritiated (3H)-propionic NHS ester (Amersham) by mixing 10 mmol Apaf-1 with 1 mCi propionic acid in 50 mM Na₂CO₃ buffer at pH 9. The resulting solution is incubated for 1 hr at room temperature and purified by size-exclusion chromatography (Nap 5, Pierce) into SuperBlock/PBS (Pierce)+1 mM DTT.

Biotinylated-cyto c is bound to streptavidin-coated scintillation beads (Amersham) by adding 100 nM protein to 10 mL of beads per 96-well plate. The beads are incubated for 20 min at room temperature, centrifuged, decanted, and then resuspended in 1 mL of SuperBlock/PBS+6% glycerol+1 mM DTT.

To optimize the concentrations of cyto c-coated beads, ³H-labeled Apaf-1, and unlabeled cyto c, a three-dimensional matrix is run wherein the concentrations of all three components are varied. Briefly, the cyto c-bound beads are serially diluted in one direction, and 10 μL of each dilution is added to each well. Next, ³H-labeled Apaf-1 is serially diluted in a second direction, and 90 μL of each dilution is added to each well. Then 1 μL of a dilution of unlabeled cyto c is added to all the wells of each plate. Thus, the wells in a single plate should contain varying concentrations of beads and Apaf-1, but the same concentration of the unlabeled cyto c. Several plates are then used to test different concentrations of the unlabeled cyto c. Finally, 2′-deoxyadenosine 5′-triphosphate (dATP) is added to each well to a final concentration of 300 μM. The mixtures in the wells are incubated for 20 min and fluorescence is detected as described below.

Compounds to be tested are suspended in DMSO to a final concentration of 100 mM. The compounds are then serially diluted by three-fold dilutions in DMSO and 1.2 μL of each dilution is then transferred to a clean, 96-well plate. The DMSO solutions are mixed with 120 μL of a solution containing the appropriate concentrations of ³H-labeled Apaf-1 and unlabeled cyto c as determined above. Ninety microliters of the resulting solution is then transferred to the clear bottom 96-well plate containing 10 μL of cyto c-coated beads at the appropriate dilution also as determined above. dATP is added to a final concentration of 300 μM and the resulting mixture is incubated for 20 min.

Luminescence is read in a Wallac Microbeta Scintillation Counter. Luminescence arises from binding of ³H-labeled Apaf-1 to the scintillant-containing beads; increase in the luminescence is due to induction of the protein-protein interaction by the compounds.

1. A method of identifying a modulator of apoptosis comprising:
(a) combining in a first mixture at least Apaf-1, cyto c, and a hydrolyzable nucleoside phosphate where each is present in a first amount that is sufficient to promote a first extent of oligomerization of at least Apaf-1 and cyto c;
(b) combining in a second mixture a test compound and two members of a set comprising Apaf-1, cyto c, and a hydrolyzable nucleoside phosphate where the two members are present in their respective first amounts;
(c) adding to the second mixture the third member of the set that was not added in step (b) in an amount less than or equal to the first amount added in step (a); and
(d) measuring the second extent of oligomerization.

2. The method of claim 1 wherein the third member of the set added in step (c) is cyto c.

3. The method of claim 1 further comprising comparing the first extent of oligomerization with the second extent of oligomerization.

4. The method of claim 3 wherein the extent of oligomerization is measured in mixtures consisting of purified components.

5. The method of claim 3 wherein the extent of oligomerization is measured by monitoring protein-protein binding.

6. The method of claim 5 wherein the extent of oligomerization is measured by monitoring Apaf-1/Apaf-1 binding.

7. The method of claim 5 wherein the extent of oligomerization is measured by monitoring Apaf-1/cyto c binding.

8. The method of claim 3 wherein the extent of oligomerization is measured by quantitating apoptosome formation.

9. The method of claim 3 wherein the extent of oligomerization is measured by monitoring the activity of caspase-9.

10. The method of claim 3 wherein the extent of oligomerization is measured by monitoring the activity of caspase-3.

11. A process comprising:
contacting a cell capable of forming an active apoptosome, the apoptosome comprising cyto c and Apaf-1, with a compound capable of decreasing the amount of cyto c necessary to form the active apoptosome, and thereby inducing apoptosis in the cell.

12. The process of claim 11 wherein the active apoptosome additionally comprises Procaspase-9.

13. The process of claim 11 additionally comprising contacting the cell with an agent to increase the level of Apaf-1 protein within the cell.

14. The process of claim 13 wherein the agent is a DNA methyltransferase inhibitor or an Apaf-1 expression vector.

15. A process comprising:
contacting a cell with a compound that promotes cyto c-dependent oligomerization of Apaf-1, thereby inducing a caspase cascade and apoptosis in the cell.

16. The process of claim 15 further comprising:
contacting the cell with an agent that increases cellular levels of Apaf-1 protein or Procaspase-9 protein.

17. The process of claim 11 or 15 wherein the cell is a human cell.

18. The process of claim 17 wherein the cell is a peripheral blood lymphocyte, a MCF I OA cell, a human mammary epithelial cell, a human umbilical vein endothelial cell, or a prostate epithelial cell.

19. The process of claim 11 or 15 wherein the cell is a cancer cell.

20. The process of claim 19 wherein the cell is a human cancer cell.

21. The process of claim 20 wherein the human cancer cell is a hematopoietic cancer cell, a skin cancer cell, a colon cancer cell, a breast cancer cell, a lung cancer cell, a renal cancer cell, a CNS cancer cell, a ovarian cancer cell or a prostate cancer cell.

22. The process of claim 21 wherein the human cancer cell is a leukemia cell, a lymphoma cell, or a melanoma cell.

23. The process of claim 20 wherein the human cancer cell is located within a solid tumor or is located on the surface of a solid tumor.

24. The process of claim 20 wherein the human cancer cell is in vitro.

25. The process of claim 24 wherein the cancer cell is a Jurkhat cell, a Molt-4 cell, a CCRF-CEM cell, a RPMI-8226 cell, a LOX IMVI cell, a BT-549 cell, a NCI/ADR-RES cell, a MDA-MB 435 cell, an HCC-2998 cell, or a NCI-H23 cell.

26. A compound having the structure (I): 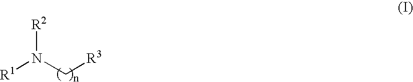
and pharmaceutically acceptable derivatives thereof;
wherein n is 0, 1 or 2;
R¹ is a moiety having the structure 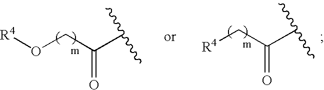
R² is hydrogen, or an aliphatic, heteroaliphatic, aryl, or heteroaryl moiety, or is —(C═O)R⁵; or R¹ and R² taken together are a cycloaliphatic, cycloheteroaliphatic, aryl or heteroaryl moiety;
R³ is an aryl or heteroaryl moiety;
each occurrence of R⁴ is independently an aliphatic, heteroaliphatic, aryl or heteroaryl moiety;
each occurrence of m is independently 0, 1 or 2; and
each occurrence of R⁵ is independently an aliphatic, heteroaliphatic, aryl, or heteroaryl moiety, or is OR⁶, NR⁶R⁷, or SR⁶, wherein each occurrence of R⁶ and R⁷ is independently hydrogen, a protecting group, or an aliphatic, heteroaliphatic, aryl or heteroaryl moiety,
whereby each of the foregoing aliphatic and heteroaliphatic moieties are independently substituted or unsubstituted, linear or branched or cyclic or acyclic, and whereby each of the foregoing cycloaliphatic, cycloheteroaliphatic, aryl and heteroaryl moieties are independently substituted or unsubstituted,
with the proviso that when R¹ and R² taken together form benzimidazole, then n can not be zero; and
with the proviso that when R¹ is the moiety having the structure 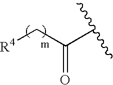
where m is 1 and R⁴ is substituted phenyl, then R² can not be hydrogen.

27. The compound of claim 26 having the structure: 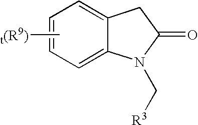
and pharmaceutically acceptable derivatives thereof,
wherein R³ is a substituted aryl or heteroaryl moiety having the structure: 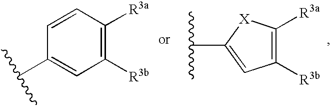
wherein X is O, S, NH, or CH₂, and each occurrence of R^{3a} and R^{3b} is independently hydrogen, halogen, substituted or unsubstituted alkyl, cyano, OR^{3c}, SR^{3c}, or NR^{3c}R^{3d}, wherein each occurrence of R^{3c} and R^{3d} is independently hydrogen, protecting group, or an aliphatic, heteroaliphatic, aryl or heteroaryl moiety; R⁹ is hydrogen, halogen, or an aliphatic, heteroaliphatic, aryl or heteroaryl moiety; and t is an integer from 0-4.

28. The compound of claim 26 having the structure: 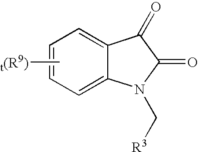
and pharmaceutically acceptable derivatives thereof,
wherein R³ is a substituted aryl or heteroaryl moiety having the structure: 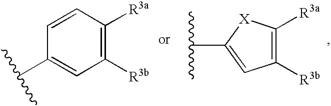
wherein X is O, S, NH, or CH₂, and each occurrence of R^{3a} and R^{3b} is independently hydrogen, halogen, substituted or unsubstituted alkyl, cyano, OR^{3c}, SR^{3c}, or NR^{3c}R^{3d}, wherein each occurrence of R^{3c} and R^{3d} is independently hydrogen, protecting group, or an aliphatic, heteroaliphatic, aryl or heteroaryl moiety; R⁹ is hydrogen, halogen, or an aliphatic, heteroaliphatic, aryl or heteroaryl moiety; and t is an integer from 0-4.

29. The compound of claim 28, wherein the compound has the structure:

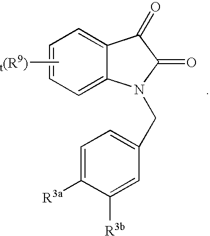

30. The compound of claim 29, wherein R^{3a} and R^{3b} are each a halogen or substituted or unsubstituted alkyl.

31. The compound of claim 29, wherein R^{3a} and R^{3b} are each Cl, Br or CF₃.

32. The compound of claim 29, wherein each occurrence of R⁹ is hydrogen.

33. The compound of claim 26 having the structure: 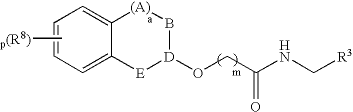
and pharmaceutically acceptable derivatives thereof,
wherein m is 0 or 1; A is —CR^{A}—, C(R^{A})₂, O, S, N, NR^{A}, or C═O; a is 0 or 1; B is —CR^{B}—, C(R^{B})₂, O, S, N, NR^{B}, or C═O; D is C or CH, E is —CR^{E}—, C(R^{E})₂, O, S, N, NR^{E}, or C═O, and A, B, D, and E are connected by a single or double bond; R⁸ is hydrogen, halogen, or an aliphatic, heteroaliphatic, aryl or heteroaryl moiety; p is 0-1; and R³ is a substituted aryl or heteroaryl moiety having the structure: 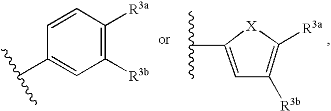
wherein X is O, S, NH, or CH₂, and each occurrence of R^{3a} and R^{3b} is independently hydrogen, halogen, substituted or unsubstituted alkyl, cyano, OR^{3c}, SR^{3c}, or NR^{3c}R^{3d}, wherein each occurrence of R^{3c} and R^{3d} is independently hydrogen, protecting group, or an aliphatic, heteroaliphatic, aryl or heteroaryl moiety.

34. The compound of claim 33, wherein the compound has the structure:

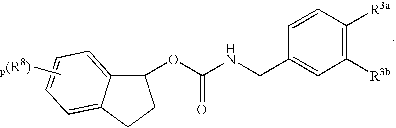

35. The compound of claim 34, wherein R^{3a} and R^{3b} are each halogen or substituted or unsubstituted alkyl.

36. The compound of claim 34, wherein R^{3a} and R^{3b} are each Cl, Br or CF₃.

37. The compound of claim 34, wherein each occurrence of R⁸ is hydrogen.

38. The compound of claim 34, wherein the compound is the S enantiomer.

39. A pharmaceutical composition comprising a compound having the structure (I): 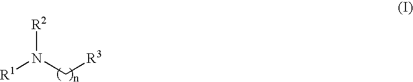
and pharmaceutically acceptable derivatives thereof;
wherein n is 0, 1 or 2;
R¹ is a moiety having the structure 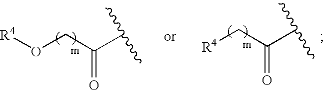
R² is hydrogen, or an aliphatic, heteroaliphatic, aryl, or heteroaryl moiety, or is (C═O)R⁵; or R¹ and R² taken together are a cycloaliphatic, cycloheteroaliphatic, aryl or heteroaryl moiety;
R³ is an aryl or heteroaryl moiety;
each occurrence of R⁴ is independently an aliphatic, heteroaliphatic, aryl or heteroaryl moiety;
each occurrence of m is independently 0, 1 or 2; and
each occurrence of R⁵ is independently an aliphatic, heteroaliphatic, aryl, or heteroaryl moiety, or is OR⁶, NR⁶R⁷, or SR⁶, wherein each occurrence of R⁶ and R⁷ is independently hydrogen, a protecting group, or an aliphatic, heteroaliphatic, aryl or heteroaryl moiety, whereby each of the foregoing aliphatic and heteroaliphatic moieties are independently substituted or unsubstituted, linear or branched or cyclic or acyclic, and whereby each of the foregoing cycloaliphatic, cycloheteroaliphatic, aryl and heteroaryl moieties are independently substituted or unsubstituted,
with the proviso that when R¹ and R² taken together form benzimidazole, then n can not be zero; and
with the proviso that when R¹ is the moiety having the structure. 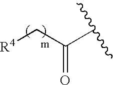
where m is 1 and R⁴ is substituted phenyl, then R² can not be hydrogen; and
a pharmaceutically acceptable carrier or diluent, and optionally further comprising an additional therapeutic agent.

40. A method for modulating apoptosis comprising: contacting cells with an amount of a compound effective to modulate apoptosis, said compound having the structure (I): 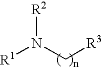
and pharmaceutically acceptable derivatives thereof;
wherein n is 0, 1 or 2;
R¹ is a moiety having the structure 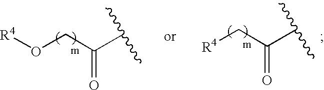
R² is hydrogen, or an aliphatic, heteroaliphatic, aryl, or heteroaryl moiety, or is (C═O)R⁵; or R¹ and R² taken together are a cycloaliphatic, cycloheteroaliphatic, aryl or heteroaryl moiety;
R³ is an aryl or heteroaryl moiety;
each occurrence of R⁴ is independently an aliphatic, heteroaliphatic, aryl or heteroaryl moiety;
each occurrence of m is independently 0, 1 or 2; and
each occurrence of R⁵ is independently an aliphatic, heteroaliphatic, aryl, or heteroaryl moiety, or is OR⁶, NR⁶R⁷, or SR⁶, wherein each occurrence of R⁶ and R⁷ is independently hydrogen, a protecting group, or an aliphatic, heteroaliphatic, aryl or heteroaryl moiety,
whereby each of the foregoing aliphatic and heteroaliphatic moieties are independently substituted or unsubstituted, linear or branched or cyclic or acyclic, and whereby each of the foregoing cycloaliphatic, cycloheteroaliphatic, aryl and heteroaryl moieties are independently substituted or unsubstituted,
with the proviso that when R¹ and R² taken together form benzimidazole, then n can not be zero; and
with the proviso that when R¹ is the moiety having the structure 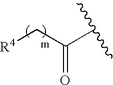
where m is 1 and R⁴ is substituted phenyl, then R² can not be hydrogen.

41. A method for treating a disorder affected by apoptosis comprising administering to a subject in need thereof an amount of a compound effective to modulate apoptosis, said compound having the structure (I): 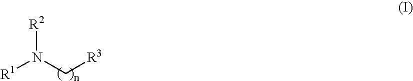
and pharmaceutically acceptable derivatives thereof;
wherein n is 0, 1 or 2;
R¹ is a moiety having the structure 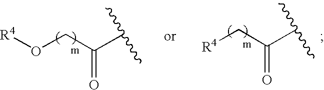
R² is hydrogen, or an aliphatic, heteroaliphatic, aryl, or heteroaryl moiety, or is (C═O)R⁵; or R¹ and R² taken together are a cycloaliphatic, cycloheteroaliphatic, aryl or heteroaryl moiety;
R³ is an aryl or heteroaryl moiety;
each occurrence of R⁴ is independently an aliphatic, heteroaliphatic, aryl or heteroaryl moiety;
each occurrence of m is independently 0, 1 or 2; and
each occurrence of R⁵ is independently an aliphatic, heteroaliphatic, aryl, or heteroaryl moiety, or is OR⁶, NR⁶R⁷, or SR⁶, wherein each occurrence of R⁶ and R⁷ is independently hydrogen, a protecting group, or an aliphatic, heteroaliphatic, aryl or heteroaryl moiety,
whereby each of the foregoing aliphatic and heteroaliphatic moieties are independently substituted or unsubstituted, linear or branched or cyclic or acyclic, and whereby each of the foregoing cycloaliphatic, cycloheteroaliphatic, aryl and heteroaryl moieties are independently substituted or unsubstituted,
with the proviso that when R¹ and R² taken together form benzimidazole, then n can not be zero; and
with the proviso that when R¹ is the moiety having the structure 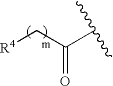
where m is 1 and R⁴ is substituted phenyl, then R² can not be hydrogen.

42. The method of claim 41, wherein the disorder is cancer.
